(19) United States
(12) Patent Application Publication
(10) Pub. No.: US 2022/0233731 A1
(43) Pub. Date: Jul. 28, 2022

(54) Device and Method for Cleaning and Disinfecting Objects, Use of the Device and the Method, and Objects Cleaned Therewith

(71) Applicant: Infinity Startup GmbH, Seligenstadt (DE)

(72) Inventors: Sing-Hong Stefan Chang, Aachen (DE); Gernot Jonathan Sümmermann, Aachen (DE)

(21) Appl. No.: 17/617,629

(22) PCT Filed: Jun. 10, 2020
(86) PCT No.: PCT/DE2020/100486

§ 371 Date: Dec. 9, 2021

(30) Foreign Application Priority Data
Jun. 10, 2019 (DE) 20 2019 103 254.5
Mar. 26, 2020 (DE) 20 2020 101 646.6
Jun. 9, 2020 (DE) 20 2020 103 326.3

(51) Int. Cl. A61L 2/10; A61L 2/20

(52) U.S. Cl. CPC A61L 2/10; A61L 2/202; A61L 2202/26; A61L 2202/17; A61L 2202/11

(57) ABSTRACT

The invention relates to a device for cleaning and disinfecting at least one object such as clothing and/or protective masks and/or personal protective equipment, in particular for the medical or laboratory sector, the device having a chamber surrounded by a housing, in which chamber at least one main body for receiving the object is arranged, characterised in that the main body has a cavity and, on its outer circumference, one or more through-flow openings leading to the cavity, wherein at least one UV radiation source emitting UV light and producing ozone is arranged in the region of at least one through-flow opening, and in that at least one air supply leads into the cavity of the main body, wherein the air can be conducted through the through-flow opening past the UV radiation source into the object. The invention further relates to a method in this respect.

### CROSS REFERENCE TO RELATED APPLICATIONS

This application is the U.S. national stage of International Application No. PCT/DE2020/100486, filed on 2020 Jun. 10. The international application claims the priority of DE 202019103254.5 filed on 2019 Jun. 10, the priority of DE 202020101646.6 filed on 2020 Mar. 26 and the priority of DE 202020103326.3 filed on 2020 Jun. 9; all applications are incorporated by reference herein in their entirety.

### BACKGROUND

The invention relates to a device and a method for cleaning and disinfecting objects, in particular for the medical and laboratory sector, according to the preamble of the 1^{st} and 18^{th} claim, as well as their use and the object cleaned therewith, and preferably relates to the cleaning and disinfection of protective clothing and protective equipment such as masks for the medical-biochemical sector. In addition, however, the method described below can advantageously also be used to disinfect articles of clothing, blankets, mattresses and other textile equipment in medical facilities, in the field of personal use or in laboratories.

According to the prior art, some disinfection processes using UV-C irradiation and ozone generation are known, but they have many disadvantages.

A method for the disinfection of liquids and gases as well as devices for the use of said method are known from the publication DE 698 01 450 T2. The publication relates in particular to methods for disinfecting liquids and gases by means of light which is irradiated into the liquids and gases through light guides. The light is used in the form of ultraviolet radiation (UVA, UVB, UVC), since this radiation is particularly suitable for killing bacteria or microscopic harmful microorganisms. Moreover, the light may be from the visible region of the spectrum. The method comprises distributing at least one light guide in an area containing the liquids or gases to be disinfected. This is followed by aligning at least one radiation unit with a high intensity light source and then irradiating the liquid or gas for a predetermined period of time. The radiation unit is in the form of a laser.

In the publication EP 2 273 004 B1 a cabinet for cleaning clothes is described, wherein the cabinet comprises a cleaning space for the clothes to be cleaned and means for generating ozone from air. The ozone is introduced into the cleaning space and brought into contact with the clothing. The cabinet has an equipment compartment in which ozone for cleaning the clothing is generated and supplied to the clothing. The equipment compartment is formed separated from the cleaning compartment by a wall.

A device for sterilizing shoes is known from the publication U.S. Pat. No. 4,981,651 A. The device comprises an elongated lamp for emitting ultraviolet radiation in a housing, as well as a heating element and a fan. The device is insertable into a shoe, wherein sterilization of the sole of the shoe is achieved by means of the lamp and the heating element. The growth of fungi and bacteria is stopped. However, only air circulation within the UV radiation source, but not in the chamber located around it, is described. An extraction by suction and a filter are not disclosed.

A similar device is described in US 2016/0339126 A1 (D1). Therein, UV sources are arranged in a housing, which are at least partially introduced into textiles to be cleaned. An extraction by suction is not provided here.

U.S. Pat. No. 3,877,152 A discloses a chamber for treating textiles having an air supply, a heat source and a UV radiation source, with which ozone is generated. A vacuum can be applied to the chamber.

Document CH 359 113 A also describes a chamber to which a vacuum is applied, thereby generating an air flow. The vacuum is used to remove the ozone and dirt particles loosened from the laundry.

A method and a device for cleaning textiles are known from DE 10 2012 209 823 A1, wherein the textiles are inserted through an opening into the chamber of a household appliance. Air is introduced into the chamber of the dryer. The air is irradiated with UV-C light, 100 to 280 nm, wherein an ozone generator provided for this purpose is arranged outside the chamber. It is not possible with this solution to efficiently clean the inner areas of the garments or the surface of other materials or objects.

Document DE 10 2007 037 984 A shows a device with an air circulation and an ozone source which can be introduced into the textiles. In this case, the air can flow through the fabric. Ozone and dirt particles are removed by a separating device. The UV source is located at the chamber end of the air supply, but the air flow is not split for multiple UV radiation sources. Furthermore, no pressure change is provided.

From the publication US 2009/0193676 A1 a device for drying shoes is known, which however separates the ozone generation and the UV irradiation from each other.

From US 2008/159907 A1, a device is known comprising a cabinet defining a chamber and wherein a drawer is slidably positioned in the chamber and movable between an open position and a closed position. The drawer defines a compartment configured to accommodate an item such as clothing (sweatshirt, shoes, jacket). In the closed position, the drawer is configured to restrict air flow from the compartment. The device includes an ozone generator to generate ozone. However, this is not positioned in the drawer position by the items to be disinfected, but in a separate machine room.

It is not possible to efficiently clean and disinfect the garments or the surface of other materials or objects, such as blankets, with the above solutions, as the penetration of the ozone cannot be completely guaranteed.

The publication DE 11 2007 000 615 T5 also describes a device for sterilizing human footwear, comprising a light source that emits radiation in a wavelength range that sterilizes the footwear by inhibiting the growth of or destroying microorganisms present in an interior region of the footwear; a support for the light source to position it to direct the radiation to the interior region of the footwear; and a light blocking device that prevents the radiation from harming a person positioned near the footwear during sterilization. Furthermore, this solution relates to a device for sterilizing human footwear having an opening into which a person's foot is inserted to stand on the footwear, comprising: a light source that emits radiation in a visible wavelength range that sterilizes the footwear by inhibiting growth of microorganisms present in an interior region of the footwear; and a support for the light source to position it to direct the radiation into the interior region of the footwear to sterilize it.

The disadvantage of these two solutions is that adhering impurities are not removed.

In an interview by Sabine Elsässer with Stefan Chang, founder of Hygenator and inventor of the RefresherBoxx, on 24 May 2018 (2018-05-24), Ref. s. URL: https://www.startupvalley.news/de/stefan-chang-hygenator-refresherboxx/ or according to XP055667315, it is stated that this box works with an intelligent and unique combination of the latest technologies (UV_C light, ozone, temperature variation, negative and positive pressure). The purely physical cleaning method denatures stench-producing bacteria such as Propionibacterium or Staphylococcus epidermis. This means that their cell walls are destroyed and the bacteria are thus killed. With the help of pressure in the box, the denatured bacteria, fungi and moisture are carried out. In addition, the RefresherBoxx has an antibacterial and antifungal effect, so that fungal infections are fought and prevented.

Furthermore, the article on the topic was published in the Aachener Nachrichten on 8 May 2018 (2018-05-08): “Aachen: Young inventor from Aachen ensures fresh shoes”, see also according to XP055663871 and under URL: https://www.aachener-nachrichten.de/lokales/aachen/junger-erfinder-aus-aachen-sorgt-fuer-frische-schuhe-aid-24508537. This only discloses the disinfection of shoes, quote “with a UV lamp you can generate ozone, the moisture is removed, but at the same time fungi and microorganisms”. Furthermore, . . . “a filter sucks up everything. The shoe is not damaged.”

How these technologies indicated in the articles interact in detail and how the box is designed, however, cannot be inferred from the two aforementioned publications.

### SUMMARY

It is the object of the invention to provide a device and a method for gentle cleaning and, in particular, practically 100% disinfection of at least one object, in particular of textiles such as clothing, protective clothing, face masks, gowns, coats, gloves and the like for the medical and laboratory sector, wherein, in particular, an environmentally friendly removal of bacteria and viruses is made possible without the use of chemical additives and the device has a simple constructive design.

In particular, it was the object to provide a device and a process to quickly reuse medical protective equipment. This reuse is of particular importance in times of epidemics and pandemics, especially when there is an acute shortage of protective equipment for logistical reasons, e.g. in poorly accessible areas and in the event of supply and production bottlenecks.

These objects are solved by a method and a device as well as their use and articles treated therewith having the features of claims 1, 18, 26, 27, 28.

Advantageous designs result from the subclaims.

### DETAILED DESCRIPTION

The device for cleaning and disinfecting at least one object such as clothing and/or protective masks, in particular for the medical or laboratory sector, has a chamber which is surrounded by a housing and in which at least one main body is arranged to receive the object, wherein the main body has a cavity and, on its outer circumference, one or more through-flow openings to the cavity, wherein at least one UV radiation source which emits UV light and generates ozone is arranged in the region of at least one through-flow opening, and in that at least one air supply leads into the cavity of the main body, wherein the air can be conducted from the cavity of the main body through the opening past the UV radiation source into the object.

Advantageously, bacteria and viruses according to Table 1 below can be removed from articles using the solution according to the invention.

TABLE 1
Blank table on treatable, in particular pathogenic microorganisms
(here examples partly with English designations)

| | Name | Name |
|---|---|---|
| Bacteria | Anthraces | Tuberculosis |
| | Diphtheria | Vibrio choleria |
| | Clostridium botulism | Pseudo monas bacteria |
| | Tetanus | Salmonella |
| | Dysentery Bacillus | Fever bacteria |
| | Cobibacillu | Bacillus typhi murium |
| | Hook-side pylon | Shigella |
| | Legionella | Staphylococcus |
| | Micro co | Streptococcus |
| Virus | Adenovirus | MERS-CoV |
| | Phagocyte cell virus | Polio virus |
| | Coxsackie virus | Rota virus |
| | ECHO virus | SARS family, e.g. Covid-19 |
| | ECHO virus 1 | Tobascco mosaic virus |
| | Influenza virus | Hepatitis B virus |
| Mold Spore | Aspergillus niger | Soft spores |
| | Aspergillus | Penicillium |
| | Dung Fungi | Penicillium chrysogenum |
| | Mucor | Other fungi penicillium |
| Fish disease | Fung 1 disease | Infectious pancreatic |
| | Leukodermia | Hemorrhagic |

The products or units listed in Table 2 below are grouped together/referred to as the object or objects:

TABLE 2
Articles to be cleaned or disinfected with the invention, in particular
materials for medical, veterinary or laboratory use

| | | Polymer-based objects, |
|---|---|---|
| | From textile materials, | e.g. made of synthetic |
| Everyday objects | such as | fibers, |
| | Synthetic fibers, | PE-based clothing and |
| | cashmere, natural | objects, polymer-based |
| | fiber, silk, velvet, wool | protective equipment, |
| | | mouth protection |
| | Made from leather | |
| | Footwear | Clothing such as |
| | | gowns, stockings |
| | | gloves, jackets, coat, |
| | | pants, |
| | Sports equipment | Jerseys, shoes, sports |
| | | shoes, |
| | | sportswear |

| | |
|---|---|
| Special | Firefighting equipment |
| Equipment | |
| | Lab coat/Laboratory protective equipment/ |
| | Laboratory equipment for biochemistry, |
| | Rescue equipment |
| | Protective equipment |
| Med. objects and | Medical protective equipment |
| devices | |
| | Respirators; |
| | Operating room supplies, critical care |
| | equipment, |

In the medical field, cleaning concerns in particular protective clothing, e.g. face masks, gloves, gowns, overcaps for head and footwear made of textile fibers or polymers according to Table 2.

Such objects or textiles and clothing from the medical or biochemical hospital, development or research area according to Table 1, in particular from safety areas, have so far often only been used once or must be cleaned at great expense with usually aggressive disinfectants in a washing machine and then dried in a tumble dryer in order to remove soiling, germs, viruses, bacteria and fungi or their spores according to Table 2.

Furthermore, in hospitals, such protective equipment is generally disinfected at high temperatures and with the use of highly reactive chemicals such as biocides, and often to the detriment of the material properties.

According to the method, the cleaning and disinfection and the elimination of pathogenic organisms according to Table 1 is carried out in or on objects according to Table 2, which consist, for example, of textile material, in particular of an article of clothing such as gowns, jackets, coats, face masks, gloves and the like for the medical or veterinary field or the laboratory field, research laboratories such as, for example, virology, vaccine development laboratories in such a way that the object is received in the chamber by means of a receiving element and UV-C light in the non-visible wavelength range and ozone are generated by means of at least one UV light source attached to the receiving element, and in that the chamber 2 is subjected to a negative pressure and/or a positive pressure, wherein an air flow is generated through the hollow receiving element by means of an air inlet leading from the housing into the chamber and an air outlet leading from the chamber out of the housing, and the air flows via outlets on the hollow receiving element and flows past ozone-generating UV lamps and is guided into the object.

Furthermore, the temperature in the chamber can be adjustable in a temperature range from −50° C. to 100° C.

By means of an air inlet leading from the housing into the chamber and an air outlet leading from the chamber out of the housing, an air flow is generated within the chamber, which flows specifically over the UV lamp via channels arranged in the receiving element and is directed to or into the object to be cleaned, in particular the object to be disinfected.

In this case, objects are preferably cleaned and disinfected which consist of textile or polymeric material or which have textile material which can also be combined with other materials such as plastic, leather, etc.

The combination of UV light and ozone, the temperature or a temperature change and the pressure or pressure change in the chamber ensure a good cleaning result and reduce or eliminate contamination with bacteria, viruses and germs/microorganisms. Preferably, the aforementioned techniques used are carried out simultaneously during a cleaning cycle. However, it is also possible to perform the selected techniques one after the other in any order or alternately.

As a means of ozone generation, a UV-C lamp is preferably used, with which ozone is generated by the radiation of UV-C light, because ions are formed by the UV-C radiation, which cause a conversion of atmospheric oxygen into ozone. The ozone is extremely reactive and has a strong germicidal effect.

The UV-C lamp is preferably dimmable (for example by means of an upstream or integrated dimmer) so that the radiation intensity of the UV-C lamp can be changed depending on the degree of soiling of the objects to be cleaned. In the case of a high degree of soiling, a high intensity of the UV-C lamp and thus more ozone is generated, and in the case of a lower degree of soiling, a lower intensity of the UV-C lamp and thus less ozone is preferably generated. The wavelength of the UV-C lamps is between 100 nm and 800 nm, preferably between 100 nm and 300 nm, particularly preferably between 150 nm and 280 nm.

In particular, the temperature modes of the device are adjustable or changeable in such a way that the temperature in the chamber increases from a low temperature to a higher temperature, or is reduced from a high temperature to a lower temperature, or that the temperature alternately increases and decreases again or decreases and increases again. The temperature can thereby be adjustable and/or variable in a temperature range from −50° C. to 100° C., preferably in a temperature range from −30° C. to 60° C., particularly preferably in a temperature range between −10° C. and 50° C. The temperature differences loosen stubborn adhesions in particular, making them easier to remove.

Furthermore, a negative pressure and/or a positive pressure can be generated in the chamber with respect to the atmospheric pressure. The pressure in the chamber may thereby increase from a low pressure to a higher pressure or be reduced from a high pressure to a low pressure. It is also possible that the pressure in the chamber alternately increases and decreases again or decreases and increases again.

The pressure in the chamber can be adjustable and/or variable in a pressure range between 0.001 bar and 10 bar, in particular between 0.1 bar and 2 bar. To achieve such a pressure difference, the chamber can be hermetically sealed inside the housing.

The pressure differences loosen dirt and other adhesions from the object to be cleaned.

In the case of a vacuum, the air is sucked out of the chamber so that pathogens, dirt particles and the like are loosened and sucked out of the chamber and out of/from the objects (e.g. shoes).

It is further possible that silver nanoparticles are introduced into the chamber from a reservoir, these may have a particle size of 1 nm to 1000 nm.

It is possible to add the silver nanoparticles to the chamber after the other techniques have been completed, so that they overlay the surface of the item, providing future protection as it has antibacterial properties, or good bactericidal activity.

Advantageously, in a cleaning cycle, heated or cooled air is introduced into the chamber and irradiated in the chamber with ultraviolet light from at least one UV-C light source. By means of the UV-C light source, ozone is generated in the chamber and thereby a disinfection and cleaning of the at least one object is realized, wherein the UV-C light source is at least partially introduced into the recesses or the cavity of the object and an air stream flows past it. A preferably alternating pressure is generated in the chamber by means of a suction device, whereby contaminations of the object are loosened and a removal of the dirt particles takes place.

In an advantageous design, the air flow can be heated by means of a heating element in the area of the intake. The heating element is preferably integrated into the channel in the form of a fan heater.

It is possible that during a cleaning cycle the temperature is reduced from 150° C. to −30° C. and then increased again or vice versa, preferably alternately within 0.5 to 66 minutes.

Furthermore, during a cleaning cycle, the pressure can be reduced from 5 bar to 0.1 bar and then increased again, preferably alternately within 0.5 to 66 minutes. Alternatively, during a cleaning cycle, the temperature may only decrease or only increase and/or the pressure may only increase or only decrease.

Particularly preferably, water vapor is added to the air flow in the area of the feed into the chamber, whereby a smoothing effect is exerted on the textiles. Ironing of the garments can then be omitted.

The steam is generated in a water tank with an integrated heating element. The use of hot steam at a temperature of 65 to 150° C. also kills bacteria, fungi and viruses present on the garment or medical item.

Preferably, a fragrance is added to the air flow and/or the chamber. The fragrance elements may be interchangeable and available in different fragrances.

All in all, it is possible to kill bacteria, viruses, fungi and their spores in or on objects with this method.

In particular, protective equipment and clothing as well as laboratory articles made of typical textile or chemical polymers and/or polymer fibers can be cleaned and disinfected in a short time, in particular within 1 to 100 minutes and preferably within 1 to 15 minutes. This is done quickly and gently, wherein the articles are immediately reusable after completion of the cleaning process.

For example, protective clothing such as face masks, protective gowns, gloves, shoes and the like, which were only intended for single use, can thus be used again or even several times without hesitation, as pathogens present have been eliminated in accordance with Table 1.

Overall, the solution according to the invention achieves a highly reliable cleaning and disinfection of the object(s). In particular, recesses and cavities present in the object or clothing can be cleaned and disinfected if the UV lamp is at least partially introduced into them. Due to the fact that the pressure differences also act in the cavities and the temperature in the chamber is cooled or heated (optionally also alternately), cavities and recesses of objects according to Tab. 2, e.g. as in the case of articles of clothing (e.g. the inside of shoes or gloves), or also of gowns and jackets and other textiles which are used in particular in the medical and laboratory sector, are also cleaned and disinfected. This allows a reliable and almost complete decontamination of the items from pathogens listed in Table 1.

It is particularly advantageous to use the method according to the invention when objects consist of or comprise textile material and cannot be washed or can only be washed to a limited extent, or when no water supply is available for washing. If objects are not or only slightly soiled and therefore washing with water is not absolutely necessary, use of the method according to the invention is also indicated for removing or disinfecting bacteria, viruses, fungi and other pathogens.

The device for cleaning and disinfecting at least one object, in particular an article of clothing or at least one recess or cavity in an object, has a chamber which is surrounded by a housing and in which the object is suspended from a main body, wherein the device comprises, according to the invention, the following means A) to D):
A) at least one UV lamp arranged in the chamber on the receiving element for generating UV-C light in the non-visible wavelength range,
B) at least one means for generating ozone,
C) at least one device for adjusting and/or varying the pressure in the chamber
D) at least one device for adjusting and/or varying the temperature in the chamber.

Furthermore, the device comprises an air supply extending from outside the housing into the interior of the chamber and comprising an air intake, and at least one heating element extending along the air supply and an air outlet extending from the chamber out of the housing.

Preferably, a fan is arranged at the air inlet and the air outlet to suck in and suck out air, respectively.

The device comprises all the aforementioned means, with which several or all of the techniques that can be carried out with them can be realized during a cleaning cycle, even in preset cycles, as required.

Advantageously, at least one UV lamp for generating (invisible) UV-C radiation and for generating ozone is arranged on the main body in the chamber. Furthermore, the device comprises the suction device for generating a negative pressure in the chamber. With this suction device, the dirt particles loosened from the object are also sucked off.

Advantageously, at least two UV lamps are arranged on the main body next to one another and spaced apart from one another, wherein the UV lamps are in particular dimmable.

In an advantageous design, the main body can be adjustable in its width and/or height. This is preferably done by means of outwardly movable or pivoting elements which can be adjusted in an infinitely variable manner or by means of rastering.

The main body is removably and/or replaceably fixed in the chamber or may be extendable from the chamber and retractable into the chamber, for example, by means of a rail system. In this way, the object can be conveniently fixed to the main body outside the chamber and then inserted into the chamber.

The cleaning cycle is advantageously adjustable by means of a control unit and the parameters such as temperature and/or pressure and/or addition of fragrances and/or silver nanoparticles are adjustable.

In an advantageous design, a fragrance element is arranged in the air supply. The fragrance element can be adjustable so that the desired intensity of the fragrance of the clothing or textile can be set.

The device preferably comprises a water tank with a heating element in such a way that water vapor can be generated and that the water vapor can be supplied to the air supply via a channel. Thus, a combined air flow of heated air and water vapor is supplied to the chamber (cleaning chamber) and introduced into the clothing.

The method according to the invention serves in particular for cleaning and disinfecting objects, in particular cavities formed by textiles and textile fabrics, wherein the textiles (or at least one textile) are inserted through an opening into a chamber formed by a housing. Preferably, in a first method step, temperature-variable air, preferably heated or cooled air, is introduced into the chamber and then the object/objects are irradiated with ultraviolet light from at least one UV source, wherein ozone is generated in the chamber by means of the UV source and disinfection and cleaning of the articles takes place. The cavity formed by at least one textile is arranged on at least one UV source in such a way that the UV source can be at least partially introduced into the objects or positioned close to the article. The germs and bacteria are killed by the combination of UV radiation and the ozone formed, whereby a disinfection of the article in question can be implemented. The cleaning effect is enhanced by the air flow, temperature and pressure in the chamber.

The generated ozone is removed by a filter placed in the area of the suction device when the cleaned air is sucked or expelled from the chamber by means of the suction device.

In the chamber, the suction device alternately generates a pressure up to a vacuum, by means of which the loosening of dirt in the objects and the removal of loosened dirt particles by means of suction take place.

The UV source operates with a wavelength of ultraviolet radiation in the non-visible UV-C range, wherein the wavelength is 100 nm and 800 nm, preferably between 100 nm and 300 nm, particularly preferably between 150 nm and 280 nm. The use of infrared radiation is also conceivable.

Preferably, air enriched with fragrances can be supplied to the chamber. In this way, the textile or object to be cleaned can be provided with a pleasant odor.

The device is used for cleaning textiles and medical protective equipment or biochemically active material, in particular objects contaminated with germs, bacteria and fungi, wherein the device comprises a housing and a chamber arranged in the interior of the housing with a main body for receiving textiles or objects. The device has an air supply, facing from outside the housing into the interior of the chamber, with at least one heating and/or cooling element extending along the air supply. At least one UV lamp is arranged on the receiving element, wherein the UV lamp is provided for generating UV radiation in such a way that ozone can be generated in the chamber. Furthermore, the device comprises a suction device for generating a negative pressure in the chamber, whereby the air and dirt particles can be suctioned or conveyed out of the chamber, wherein the generated/used ozone is removed by a filter in such a way that cleaned air is ejected.

In an advantageous design, the suction device is dimensioned in such a way that it can generate a vacuum in the chamber. This makes it easier to remove dirt and germs from the textiles, especially if an alternating vacuum is generated.

Preferably, a silver nanoparticle and/or fragrance delivery system is provided on the device. The silver nanoparticles are deposited in the articles and serve to prevent the growth of viruses and bacteria in the cleaned articles.

In this process, the silver nanoparticles and/or fragrances are placed in the chamber. Preferably, the silver nanoparticles and/or fragrances are then introduced directly into the cavity/cavities formed by the object(s). Preferably, this is done via the air supply.

The air that can be introduced into the chamber can be heated by means of a heating coil arranged around the air supply or cooled by means of a cooling device. A fan is preferably arranged at the end of the air supply on the housing side, which sucks in the air and conveys it into the chamber. A combined fan heater may also be used. Extraction is carried out in a similar manner, wherein a second fan extracts air from the chamber and releases it into the environment.

It is also possible that a heating and/or cooling device is arranged in the chamber and the supplied air is not heated or cooled.

Preferably, the cleaning cycle is adjustable by means of a control unit, wherein individual or all parameters are adjustable. The parameters include the temperature and/or the pressure within the chamber and, if desired, the addition of fragrances and/or the silver nanoparticles. The chamber may further find application for drying the objects. The device uses as UV source the spectrum of the UV-C light, wherein the combination of UV radiation, ozone, temperature programs and vacuum air change application eliminate bacteria, viruses and fungi. The parameters can be determined independently by the device by inputting properties of the object to be cleaned. These properties include, for example, the type of material, degree of soiling, degree of moisture or the like.

The adjustable temperature programs include the possibility of heated or also strongly cooled air, which is led to the cleaning locations to be applied. The ozone is synthesized directly at the UV-C source, the UV lamp, i.e. synthesized and applied at the desired application area.

The vacuum-air change system removes dead and possibly still organisms from the textiles.

The chamber has an antiviral, antibacterial and antifungal effect during the cleaning cycle and cleans the products reliably, starting from the inner area where the UV lamps are located. The air with the ozone penetrates the textiles, ensuring extremely reliable cleaning and disinfection. The disinfection and cleaning occur without the addition of chemicals, which results in a high degree of environmental friendliness and material protection. Due to the use of steam, only a small amount of water is required for cleaning or smoothing the items.

Preferably, the items such as jackets, gowns, gloves, shoes, face masks for the medical and laboratory area are cleaned with. However, it is also possible to reliably disinfect other items for medical and laboratory facilities.

The device and the chamber disposed therein are configured such that objects can easily fit therein and be arranged or hung therein without creasing. The dimensions in terms of width, height and depth are preferably 30/125/60 cm.

An increase in the dimensions, e.g. to an apparatus volume of 2 to 5 m³ with correspondingly more UV-C or ozone sources with greater pumping and heating power or larger components, has no influence on the effectiveness of the mode of operation. Preferably, the device should not fall below the dimensions mentioned.

The device according to the invention is effective against virtually all pathogens, such as viruses and bacteria, as well as various types of mites, such as house mites and scabies mites, and against fungi in the form of athlete's foot or skin fungus according to Table 1

Furthermore, irradiation of the objects and clothing with ultraviolet light and/or application of ozone inactivates or destroys organisms. This can be considered as disinfection of the irradiated area. These microscopic organisms include, for example, spore-forming and non-spore-forming life forms or viruses or bacteriophages or cysts according to Table 1.

The device according to the invention is used in particular for the disinfection/killing/elimination of viruses, especially Covid-19 Corona viruses or viruses of the SARS family and the microorganisms listed in Table 1, in particular for work clothing or also disposable clothing in laboratories and the medical sector.

For this purpose, UV light with a wavelength range of 185 nm to 400 nm, ozone, a temperature of 20-100° C., also alternating, a (negative) pressure of 0.1 bar to 5 bar is generated in the device and the objects (e.g. the clothing) are exposed to it.

It is thus possible that air is blown into the main body at pressure via a corresponding pump, which then flows through the main body via the through-flow openings past the UV-C lamps against or into the objects to be cleaned, preferably textiles, and is then discharged into the ambient air via a filter and an air outlet.

Further flow openings (without UV-C lamps) can be arranged in the main body, through which air flows out against the objects to be cleaned. Then, if desired, an additional drying process is also achieved.

Furthermore, it is possible that air is sucked out of the cavity of the main body via a corresponding pump/vacuum pump, the direction of flow is then opposite. If the pump is connected to the main body and air is sucked out of it (and a vacuum is created), the air is sucked out of the chamber via the objects held on the main body and the UV-C lamps into the cavity of the main body and then conveyed to the outside. As a result, particles (e.g. dirt, pathogens, killed) located in the device and on the objects to be cleaned are sucked out.

The positive pressure and the negative pressure can be generated by one or two separate pumps. Furthermore, in an advantageous embodiment, the device is also suitable for mobile use.

It is also possible to allow water vapor or solvent vapors, liquid or gaseous fluid—which contain solvents—to flow through the main bodies, whereby, for example, stains, dirt or impurities can be removed.

Furthermore, water vapor with overpressure and/or underpressure can be led through the main bodies and via the flow openings to the objects to be cleaned.

Also, the water vapor may optionally be mixed with a solvent against stains. Negative pressure can effectively remove stains or other impurities, positive and negative pressure.

By flowing water vapor, solvent vapors, and optionally other suitable fluids, preferably with alternating pressure, especially negative and positive pressure, stains and other contaminants can be effectively removed.

Due to the fact that heated/cooled air in the aforementioned temperature ranges is fed into the chamber by means of the air supply, or the air in the chamber is heated or cooled, that the UVC radiation sources are introduced into the objects or are positioned close to them, a better penetration of the objects with the ozone via the air flow introduced directly into the objects and acting on them is ensured than with the prior art and thus a better cleaning effect or disinfection effect and disinfection of bacteria and viruses are thus achieved.

This effect is further improved if the main bodies for the objects to be cleaned are connected to a chamber-side end of the air supply, and a division of the air flow within the chamber takes place with the main body through which the air flow is directed.

The hollow main body may, for example, comprise or be composed of pipes, pipe sections and/or pipe connecting elements. The projections adjoining the hollow, e.g. tubular, main body and/or the additional openings can thereby be formed, for example, from pipe fittings, pipe bends, cross pieces, pipe nipples and the like.

The UV lamps preferably have an external thread which corresponds to the internal thread of the pipes, pipe bends, cross pieces, pipe nipples.

The tubular main body can be screwed, glued, connected by means of press connectors with the pipe bends, cross pieces, pipe nipples and the like.

The tubular main body as well as the screw connection elements and pipe connection elements and the like are made, for example, of gunmetal, brass, stainless steel or malleable cast iron or can also be made of plastic.

The tubular main body and the fitting elements or the flow-through openings or projections in a main body have a connection thread of ¾ inch to 1¼ inch.

Preferably, the thread for screwing in the UVC light source is a 1¼ inch internal thread. The thread of common UVC lamps fits exactly into the 1¼ inch thread. In this case, the pipes, pipe connecting elements, fittings and the like preferably have an outer diameter of 32 mm.

Alternatively, the hollow main body also consists of only a single piece and is manufactured by means of 3D printing or, for example, by injection molding.

In addition to the openings in the main body in the area of the UV lamps, there may be further flow openings.

According to the invention, the device thus finds application for killing bacteria, viruses and fungi and other pathogens on or in objects, in particular articles of clothing or protective clothing, protective equipment, for the medical, veterinary or laboratory sector and particularly preferably objects in the medical, veterinary or laboratory sector. Of course, it is also possible to clean the professional clothing of other professions, e.g. nursing professions or professions that come into close contact with others. But also in the hotel sector for the cleaning of the clothes of the staff or the guests or also in the private area, e.g. if endangered persons are in the household, the device can be used for the cleaning and disinfection of clothes and/or other objects.

Advantageously, the objects, in particular medical devices and articles and medical protective clothing, have on their surfaces at the end of each treatment only residues of >less than 10%, preferably less than or equal to 1%, in particular less than or equal to 0.1%, of biochemically biologically active organisms and/or biochemical materials.

Objects that are normally intended for single use only (disposable respiratory masks, protective gloves, gowns and the like) are thus reusable after treatment.

### BRIEF DESCRIPTION OF THE DRAWINGS

The invention is explained in more detail below with reference to an exemplary embodiment and associated drawings, wherein:

FIG. 1 shows a device according to FIGS. 2 and 3 in closed position,

FIG. 2 shows a side view of a device having first and second base element within the chamber,

FIG. 3 shows a three-dimensional representation according to FIG. 2,

FIG. 4 shows a representation similar to FIG. 6 but without a lid in the insertion and instead with cross-struts 12 between the sides S1 and S2,

FIG. 5 shows a further structure of a device according to the invention in 3D view,

FIG. 6 in front view and

FIG. 7 in side view.

FIG. 8 shows a front view of a shoe adapter,

FIG. 9 shows a 3D view of a shoe adapter,

FIG. 10 shows a front view of a clothing adapter,

FIG. 11 shows a 3D view of a clothing adapter,

FIG. 12 shows two half shells that can be connected to each other,

FIG. 13 shows a half shell which is closed with a plate,

FIG. 14 shows a device in the form of a box with adapter inserted in the longitudinal direction,

FIG. 15 shows the box with the adapter pulled out,

FIG. 16 shows a box with adapter received transversely in the chamber,

FIG. 17 shows a round rotatable main body with a plurality of projections in side view,

FIG. 18 shows a round main body according to FIG. 20 in plan view,

FIG. 19 shows the main body according to FIG. 20 in a box RB,

FIG. 20 shows a device with several tubular main bodies which extend vertically next to one another and are connected to one another,

FIG. 21 shows the front view according to FIG. 20,

FIG. 22 shows the device according to FIGS. 20 and 21 from another perspective with an exemplary laboratory coat positioned above the main body,

FIG. 23 shows a device with tubular main bodies, in which a plurality of upwardly extending projections are arranged next to and above one another,

FIG. 24 shows the front view according to FIG. 23,

FIG. 25 shows a device with a cross-shaped hollow main body with three projections facing upwards,

FIG. 26 shows a partial view of the device according to FIG. 25 with objects to be cleaned held above it,

FIG. 27 shows a main body extending substantially horizontally with three upwardly facing through-flow openings.1

### DETAILED DESCRIPTION OF THE PREFERRED EMBODIMENTS

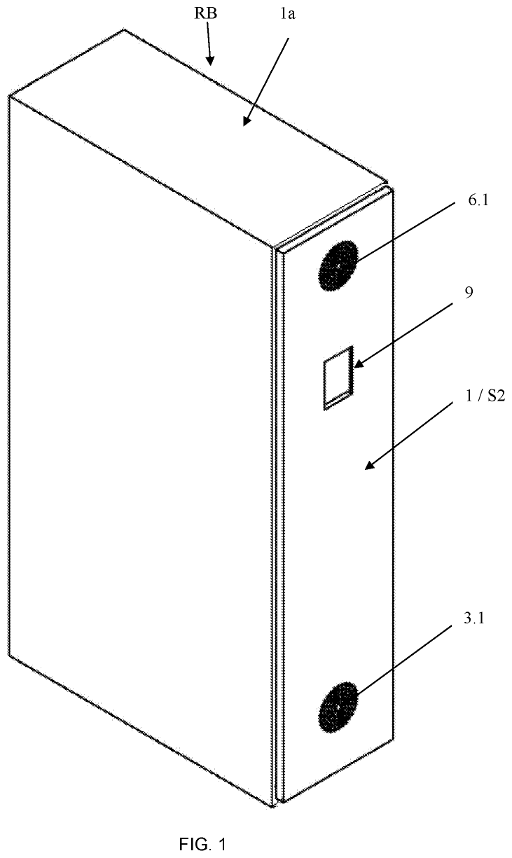

FIGS. 1 to 3 show a schematic structure of the device for cleaning and disinfecting objects, in particular for cleaning and disinfecting the interior of objects such as medical equipment, shoes, helmets, gloves, sports equipment, T-shirts, shirts, suits (garments) and bags. The device comprises a box RB having a chamber 2/cleaning chamber formed herein in a housing 1, wherein the housing 1 is insertable into and withdrawable from a cabinet body 1a of the box RB enclosing the housing 1 via a rail system (not shown). The cabinet body 1a is open in the direction of the housing 1 (via a vertically extending undesignated opening), so that the housing 1 can be pushed into it. Otherwise, the cabinet body is closed off at the bottom, top, sides and rear. The housing 1 with the chamber 2 (cleaning chamber), which can be pushed into the cabinet body 1a, is preferably frame-like in this case. The housing 1 has a base B and a lid D, wherein the base B and the lid D are connected to one another via two end-side vertical and mutually parallel side parts S1, S2. The side part S1 is arranged in the direction towards the cabinet body 1a and the side part S2 is arranged opposite thereto. The side part S2 closes the cabinet body 1a in the inserted state. (See FIG. 1)

The device has in the lower region an air supply 3 in the form of a channel, wherein the housing 1 has at its front an opening in the form of an air inlet 3.1 for the air supply 3 leading into the chamber 2, wherein a fan 3.2 is arranged at the housing-side end of the air supply 3, which forms the air inlet 3.1. The fan 3.2 is designed in such a way that fresh air is drawn in from the environment and can be conveyed through the air supply 3 into the chamber 2.

The air supply 3 is preferably of tubular design and has an angle at its chamber-side end 3.3, preferably of 90°. The angle acts as a guide for the air, whereby a central outflow of the aspirated air in the chamber 2 can be achieved.

Two hollow-shaped main bodies 4 are provided, both of which are connected to the chamber-side end of the air supply and extend upwards into the chamber 2.

Each main body 4 has an air guide by means of which the air flow guided through the end on the chamber side can be divided into a first and second air flow. For each air flow, each main body has a UV radiation source arranged at an outlet opening of the air flow.

Depending on the design of the main body, the outlet openings are arranged radially or, in the example shown, axially with respect to the flow of air.

The wavelength of the ultraviolet radiation is preferably between 100 and 300 nm. The ultraviolet radiation is so short-wave and energy-rich that it is absorbed by molecular oxygen (O₂). In this process, the molecular oxygen (O₂) is split into two free oxygen radicals (2 O°), each of which reacts further with another molecule of oxygen (O₂) to form ozone (O₃).

The ozone thus formed at the UV lamp 5 disinfects the objects against germs, fungi and bacteria.

Furthermore, the chamber 2 (main chamber or cleaning chamber) comprises an exhaust device 6 in the ceiling area of the chamber 2. The suction device 6 is preferably arranged in the upper region of the device, here at the top in the side part S2, and comprises, for example, an air outlet 6.1 leading outwards through the housing 1 at its front, in which a second fan 6.2 is mounted. The fan 6.2 is configured to draw air out of the chamber 2, thereby creating a negative pressure. An alternating pressure is achieved in the chamber 2, and the suction device 6 may be configured such that a vacuum can also be generated in the chamber 2. The vacuum-air alternating system removes dead, living and eliminated organisms from the objects. The suction device 6 with the air outlet 6.1 may be preceded by a replaceable filter, which is not shown, so that the polluted air can be cleaned and the ozone removed before it exits the chamber.

For more effective cleaning, the air in the region of the air supply 3 is heated or cooled or alternately heated and cooled. For this method step, a heating device 3.4 is arranged in the region of the air supply 3, which is preferably wound around the air supply 3 in the form of a heating coil. Alternatively, a fan heater can also be used as a combination of heating device 3.4 and fan 3.2. The air flowing through the air supply 3 is heated in the region of the heating device 3.4. Alternatively or additionally, a cooling element 6.1 can be provided in the area of the air supply 3.

It is also possible to heat or cool a heating or cooling element in the cleaning chamber 2 and accordingly the interior of the chamber.

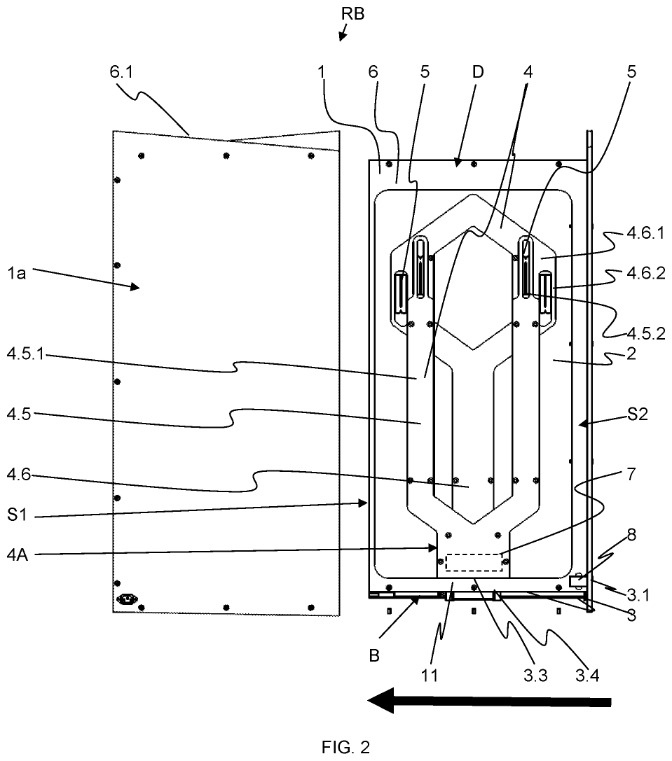

In an advantageous design, according to FIGS. 1 and 2, a water tank 7 with a heating element 7.1 arranged therein is provided in the base region of the device. The water is heated in such a way that water vapor is produced, wherein the water vapor is supplied to the air supply 3 via a conduit 7.2.

Mixing of heated air and the generated water vapor takes place, wherein the mixture is fed to chamber 2 via the chamber-side end 3.3.

The garment to be cleaned is smoothed by the moisture-enriched air, which eliminates the need for subsequent ironing. The water tank 7 is advantageously designed to be filled via a side opening or to be completely removable.

It is also possible for the base B to be double walled and for air to be directed into the chamber 2 from the air inlet 3.3 via the air supply 3 through the base-side cavity formed as a result. Similarly, water vapor may also flow through the base-side cavity into the chamber 2 via the air inlet 3 or another inlet (not shown).

The fan 3.2, the heating device 3.4, the water tank 7 and the heating element 7.1 are then also arranged in the base-side cavity.

In addition, a fragrance capsule 8 may be insertable into the air supply 3 to produce a fresh scent of the objects. The released fragrances may be added with the air drawn in by the air supply 3 and directed into the chamber 2. Preferably, the fragrance intensity is adjustable via a manual controller 8.1 or via the control unit 9 with a front display of the device.

The device also offers the possibility to gently dry damp objects in a short time.

With the device according to the invention, an environmentally friendly cleaning and disinfection of the objects is possible without the addition of chemical cleaning agents. Viruses, bacteria, germs and fungi and their spores according to Table 1 and other pathogens are effectively eliminated.

In FIG. 1, the device is shown in the closed state. The insert in the form of the housing 1 is inserted into the cabinet body 1a of the box RB, with the side part S2 closing off the cabinet body 1a. A seal may be provided between the side part 1 and the cabinet body 1a.

The side part S2 contains the display 9, the air inlet 3.1 and the air outlet 6.1. Alternatively, the air inlet 3.1 and the air outlet may also be located in the rear side part S1 (not shown), in which case the cabinet body 1a should be open at its rear side, which is not designated, at least in these areas.

The device is operated by means of the display 9 or also via an app.

It is possible to arrange one or more main bodies in the chamber 2, so that one or more objects can be disinfected and refreshed simultaneously.

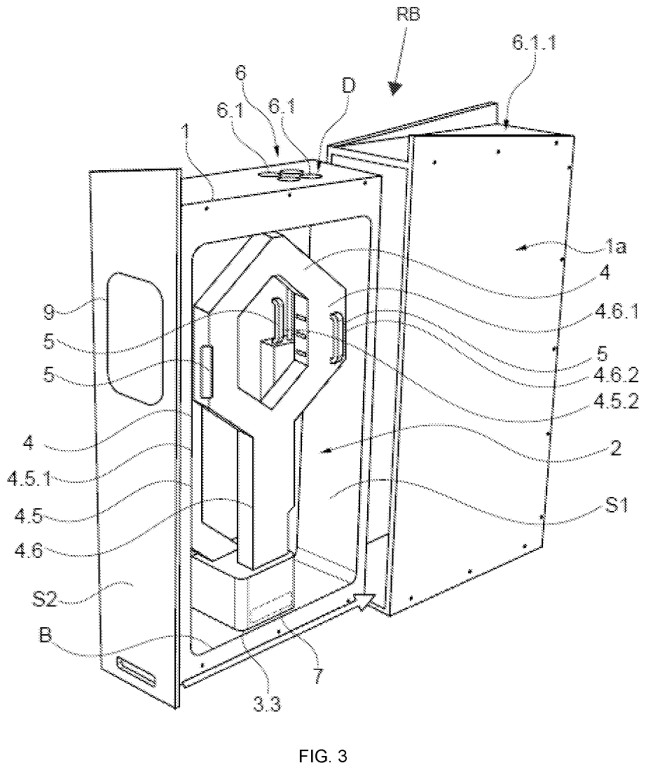

According to FIGS. 2 and 3, the housing 1 of the box RB has a chamber 2 (main chamber or cleaning chamber) with an air supply 3 with an air inlet 3.1 arranged in the front and an alternatively designed main body 4. The main body 4, which is of hollow construction, is directly connected at its underside to the air supply 3 and extends upwards in a branching configuration and is in the form of a first adapter 4.5 and a second adapter 4.6, which extend upwards into the chamber 2. The first and second adapters 4.5, 4.6 of the main body 4 are of course also hollow and have an air guide 4.5.1, 4.6.1, by means of which the air flow guided through the chamber-side end 3.3 to each adapter 4.5, 4.6 can be divided and thus leads in a first air flow into the first adapter 4.5 and in a second air flow into the second adapter 4.6.

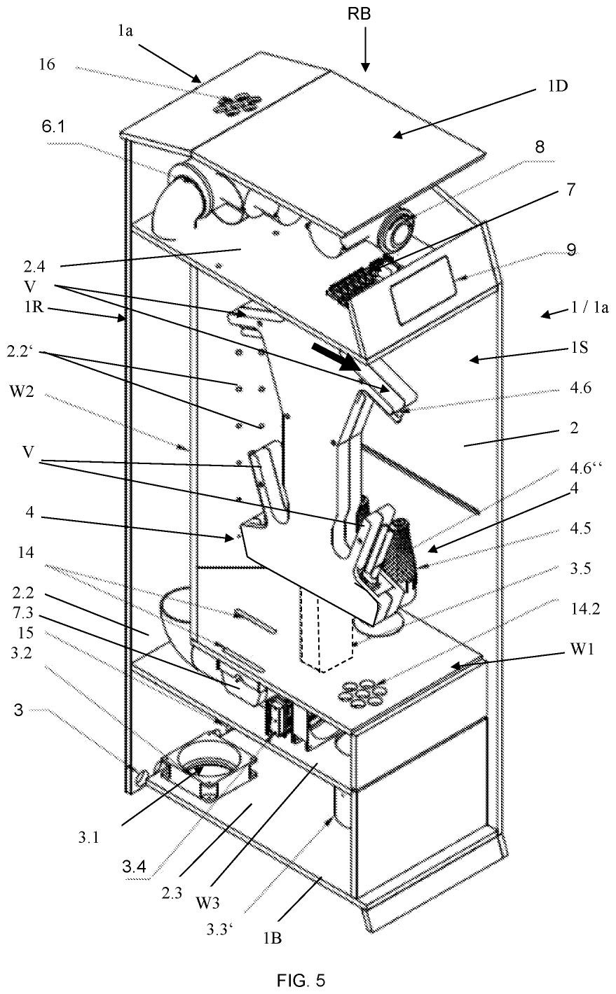

The first adapter 4.5 and the second adapter 4.6 each have two branches. In the area of each branch at least one outlet opening 4.5.2, 4.6.2 is provided, according to FIG. 2 a total of four outlet openings (two for each adapter 4.5, 4.6), in the area of each of which a UV radiation source 5 is arranged. The outlet openings can be arranged radially or axially with respect to the individual air flow. In the present example according to FIG. 5, the first adapter is of Y-shaped (Y) design and has a first and second UV radiation source 5 at its chamber-side end. Such an adapter is particularly suitable for positioning objects forming a cavity in the form of shoes, gloves, breathing masks, helmets and the like, as these are placed or plugged onto a single UV radiation source 5 or two UV radiation sources 5 in a spanning manner.

The second adapter 4.6 differs in its shape from the first adapter 4.5 in such a way that the two air flows or air channels are brought together again, or are connected to each other. The shape of the second adapter 4.6 corresponds to a “koppa” (o Greek alphabet). The UV radiation sources 5 are arranged in the region of the two divided air streams. With such a design of an adapter 4.6 textile objects can be hung up without a further hanger.

In the chamber 2 (main chamber or cleaning chamber), a main body with preferably two adapters is thus arranged here, wherein two adapters of the first adapter type 4.5, two adapters of the second adapter type 4.6 or one adapter each of the first type 4.5 and the second type 4.6 are used. Preferably, in the first and second adapters 4.5, 4.6 the wirings for the connection of the UV radiation sources 5 are provided.

The adapters 4.5, 4.6 may have symmetrically and/or asymmetrically distributed holes and/or passages throughout for distributing the air or water vapor.

Above the area of the air supply 3.3 arranged on the chamber side in the base B, a vertically extending area of an air channel 4A is provided. Adjacent to its upper end are the first and second adapters 4.5 and 4.6 of the main body 4, which are of channel-like design and through which the air flow is further divided.

It is possible that the adapters 4.5 and 4.6 are formed integrally with the air channel 4A and are removably and interchangeably attached to the base, or that the adapters 4.5 and 4.6 are formed separately and are removably attached to the upper end of the air channel 4A.

In any case, for example, only one or two or more first adapters 4.5 or one or two or more second adapters or a different number and combination of adapters may be used, depending on the object to be cleaned.

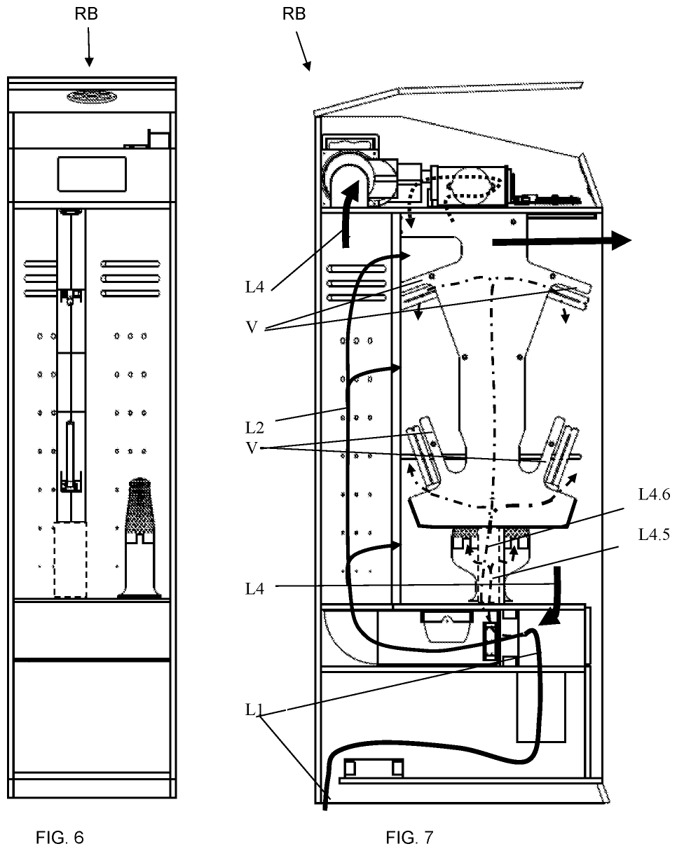

According to FIGS. 5 and 6, the water tank 7 of the box RB with a heating element not shown arranged therein is arranged in the region of the transition between the chamber-side end 3.3 of the air supply and the first and second adapters 4.5, 4.6, here in the region of the air channel 4A. The water is heated in such a way that water vapor is formed, wherein the water vapor is supplied to the air supply 3 via an undesignated conduit.

The heated air and the generated water vapor are mixed, and the mixture is fed via the chamber end 3.3 to the cavities formed in the adapters and is conducted via these to the UVC radiation sources.

The air enriched with moisture smooths the object to be cleaned, eliminating the need for subsequent ironing.

The additional use of a special ironing liquid is also possible, so that instead of water the ironing liquid is filled into the schematically indicated water tank 7.

The smoothing of the objects by means of steam is preferably carried out after the UV radiation sources have been switched off and thus after the object has been treated with UV radiation.

For more effective cleaning, the air in the area of the air supply 3 is heated or cooled as already described or alternately heated and cooled. For this purpose, the heating device 3.4 is arranged in the area of the air supply 3. Alternatively, a fan heater can also be used as a combination of heating device 3.4 and fan 3.2. The air flowing through the air supply 3 is heated or cooled in the area of the heating device 3.4. The temperature is adjustable and/or variable in a temperature range from −50° C. to 100° C., preferably in a temperature range from −30° C. to 60° C., particularly preferably in a temperature range between −10° C. and 50° C.

In a further design, a fragrance capsule 8 is arranged in the region of the air supply 3 for generating a fresh fragrance of the objects into the air supply 3. The released fragrances can be added with the air sucked in by the air supply 3 and fed into the chamber 2 (main chamber or cleaning chamber).

According to the preceding figures, the chamber 2 (main chamber or cleaning chamber) according to the exemplary embodiment in FIGS. 5 and 6 comprises a suction device 6 in the ceiling area of the chamber 2. The suction device 6 is preferably arranged in the upper region of the device and has an air outlet 6.1 directed upwards and outwards, in which likewise a fan (not shown) is provided. The suction device 6 can also be supplemented with a replaceable filter, not shown, so that the polluted air can be cleaned and the ozone removed before it exits the chamber. The filter is preferably arranged in the area of the fan and the suction device 6.

Also according to FIGS. 2 and 3, an alternating pressure can be achieved in the chamber 2 with the suction device 6 and the air supply 3, wherein the suction device 6 can be designed in such a way that a vacuum can also be generated in the chamber 2. For such a design, the chamber 2 is preferably air-tight.

From FIG. 3 it can be seen that the suction device 6 with one or more air outlets 6.1 can also be arranged in the lid D. In this case, there are outlet openings 6.1.1 in the undesignated upper side of the cabinet body 1a, through which the extracted air can escape. In the inserted state of the housing 1, the outlet openings 6.1.1 of the cabinet body 1a are located above the air outlets 6.1 in the lid D of the housing 1.

Furthermore, it is possible to arrange an additional pressure system 11 in the area of the base B of the chamber 2, by means of which the air supply 3 and the suction device 6 are supported to generate a negative or positive pressure.

The pressure in the chamber 2 can be adjustable and/or variable in a pressure range between 0.001 bar and 10 bar, in particular between 0.1 bar and 2 bar.

The use of a filter is particularly recommended as even low concentrations of escaping ozone are harmful to health due to irritation of the respiratory tract.

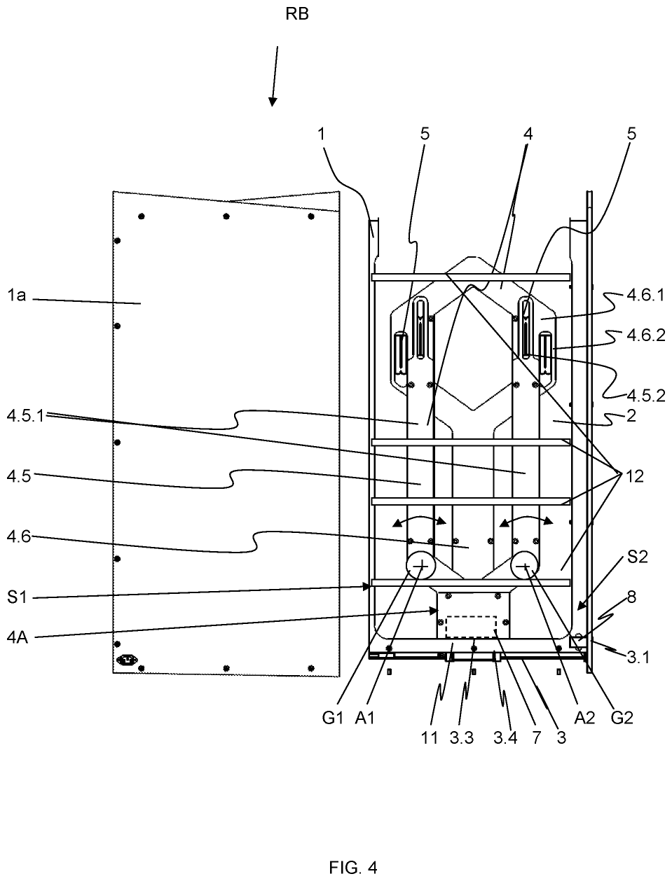

FIG. 4 shows a representation of a box RB, similar to FIG. 6 but without a lid in the insertion and instead with cross-struts 12 between the sides S1 and S2, which stabilize the sides S1, S2 relative to each other and ensure that the objects to be cleaned do not protrude laterally from the housing 1 and prevent them from being inserted into the body. It is possible here to insert the objects to be cleaned from above.

The suction device (not shown here) is then arranged in the front side part S2 as in FIG. 1 or also in the rear side part S2, in which case corresponding air outlets are then present in the cabinet body 1a in the rear wall.

In this variant, it is indicated that the first and second adapters 4.5 and 4.6 are detachably connected to the air channel 4A of the main body 4.

Instead of cross-struts, one or more plate-like elements may extend between side parts S1, S2.

Such cross-struts or plate-like elements can also be used in the variants described above.

Furthermore, it is possible according to FIG. 7 that, for example, the first adapter 4.5 has a joint G1 and G2 in each of the two upwardly extending regions of the air guides 4.5.1. The region shown here extending vertically upwards, for example in an initial position, is thereby designed to be pivotable about a horizontal axis A1, A2, which is indicated by the double arrows. As a result, the upper ends of the two air guides 4.5.1 of the adapter 5.5 can be pivoted towards and away from each other, and thus their distance can be adapted to the objects to be accommodated.

With the device according to the invention, the air flow supplied to the chamber 2 (main chamber or cleaning chamber) is divided in the chamber via the channels formed by the adapter elements and is guided in each case to a UV radiation source 5.

The number of channels formed in an adapter corresponds to the number of UV radiation sources 5 provided on the adapter.

It is thus also possible to provide a main body 4 or adapter not only with two, but also with three, four or more upwardly extending branches, each of which guides the air flow to a UV radiation source 5.

Preferably, the UV radiation sources 5 are located at the air outlet areas of the adapters.

The adapters also form the receptacles for the objects to be cleaned.

The adapters can be designed in such a way that the distance between the upper areas that receive the objects to be cleaned can be changed.

It is possible to arrange several of the substantially parallelepipedal devices according to the invention side by side and/or one above the other in a battery-like manner. The devices can also be connected to each other.

Furthermore, it is also alternatively possible to provide a cabinet body with a pivotable or laterally displaceable door, in which case the chamber/cleaning chamber is formed in the cabinet body.

Depending on the object to be cleaned, the individual parameters such as pressure, temperature, UV radiation and ozone can be set. For example, when cleaning leather, it is advisable to use the maximum power of the device, while cashmere, for example, should only be treated gently.

In FIGS. 5 to 7, a further variant of a device according to the invention is illustrated. Here, no housing 1 is provided which can be pushed into a cabinet body 1a of the box RB, but the housing 1 forms the cabinet body 1a. The chamber 2 (main chamber or cleaning chamber) is thus not withdrawable here, but is formed in the cabinet body 1a. The device thus has a body 1a in which the chamber 2 (cleaning chamber) is formed. Here, two main bodies 4 are provided in the chamber 2 for receiving objects, namely a first main body 4.5 for accommodating a pair of shoes or a pair of gloves or protective masks and a second main body 4.6 for accommodating clothing in the form of shirts, gowns, jackets and the like. The first and second main bodies 4.5, 4.6 are hollow and have an air supply in their interior leading through the hollow structure which is not visible. The air stream flowing in via a conduit can be divided into a first and a second air stream for each adapter 4.5, 4.6 and flows past the UV lamps of the adapters 4.5, 4.6 which are not visible here and are not designated. These UV lamps are arranged at outlet regions of the adapters 4.5, 4.6, so that the air flow emerging from the adapters 4.5, 4.6 flows past the UV lamps.

The first main body 4.5 branches upwards into two unnamed adapter elements with outflow openings (not shown) in which UV-C lamps are arranged and has a size which allows a pair of shoes, gloves, protective masks and the like to be positioned on it, so that the UV lamps are at least partially located in the objects to be cleaned and the air flow emerging from the first adapter 4.5 penetrates the object with the ozone formed by the UV lamp and possibly also penetrates it (in the case of textile material).

The second main body 4.6 is also of hollow design and has a bow-shaped design in its upper region, so that a shirt, a jacket, a smock, items of Table 2 and the like can be hung up above it. For this purpose, two projections V facing laterally away from each other and angled downwards with undesignated openings extend on the upper region of the second main body 4.4, in which the UV-C lamps are arranged.

Preferably the second main body 4.6 can be pulled out of the device in the direction of the bold arrow, for example, via an upper rail-like suspension 13 (see FIG. 10), so that the clothing or other objects can be better positioned above it. At opposite projections V of the hanger-shaped region there are undesignated outlet openings, and within each end there is a UV lamp. After receiving the objects, the second adapter 4.6 is pushed back into the device. During cleaning operations, the air flow is directed into the second hollow shaped adapter 4.6 either from above or from below, flows past undisclosed UV lamps and out of the outlet openings in the bracket shaped area. Two upwardly facing projections V are additionally provided on the lower region of the second main body 4.6. These have lateral slots 4.6″ through which ozone-enriched air can flow into the chamber 2. In the case of longer items of clothing, this also ensures cleaning of the lower region if these reach over the lower projections V.

A second chamber 2.2 extends below the chamber 2 and laterally thereof in the body 1a. The second chamber 2.2 is separated from the second chamber 2.2 by a horizontal wall W1 and a vertical wall W2. Below the second chamber 2.2 a third chamber 2.3 is arranged, which is separated from the second chamber 2.2 by a horizontal wall W3. Above the first chamber 2 and the vertical region of the second chamber 2.2 is a fourth chamber 2.4 separated therefrom by a horizontal wall W4. Below the third chamber, the cabinet body has a base 1B, a rear wall 1R, a lid 1d, two side walls 1S (only one of which is visible) and a door (not shown here) arranged below the display 9.

In order to be able to represent the interior design of the device, a side wall and the door were not drawn in.

Laterally in the lower area of the third chamber 2.3 there are recesses 3 for the air supply. In the lower third chamber 2.3 there is further provided an air supply 3.1 in the base 1B and an inlet pump or fan 3.2. An inlet regulation 3.3′ leads to the second chamber 2.2 from the third chamber 2.3 and via this the air supply, not designated here, into the second chamber 2.2. The air is heated in the second chamber 2.2 by means of a heating element 3.4 to the desired/preset temperature. The air flows in the lateral area of the second chamber 2.2 upwards into the fourth chamber 2.4 and in the vertical separation W3 between the chamber 2 and the second chamber 2.2 there are apertures 2.2′ through which a part of the sucked air can enter the chamber 2.

The aspirated air is guided into the interior of the main bodies 4.5, 4.6. This is carried out either from below from the second chamber 2.2 via a supply line 3.5 (shown here dashed) or from above from the fourth chamber 2.4 via a supply line not shown here. In the base or the horizontal wall W3 of the second chamber there are openings 14 through which water vapor can be supplied for smoothing the objects. This is preferably supplied under pressure from an evaporator 7.3 provided for this purpose. Water is supplied to the evaporator 7.3 from a water tank 7, which is arranged here in the upper fourth chamber 2.4 and is connected to the evaporator 7.3 via a conduit not shown. The hot water vapor additionally serves to kill viruses, bacteria, fungi and other germs.

Furthermore, openings 14.2 may be provided in the bottom (wall W2) of the chamber 2 for generating a circulating air between the chamber 2 and the second chamber 2.2. Furthermore, a connection 15 for a vacuum pump is provided in the third chamber 2.3, by means of which a vacuum can be generated in the chamber 2. The vacuum is formed in the chamber 2 and in the second chamber 2.2.

As in the example described above, a fragrance capsule 8 is also provided, here arranged in the fourth chamber 2.4 (optional).

The electronics are also located in the fourth chamber 2.4 and the control unit 9 with a corresponding operating display is provided on the front side thereof. The lid 1D of the fourth chamber 2.4 is preferably pivotable upwards. Openings 16 are arranged in the lid 1D, which serve to cool the electronics and in/under which one or more fans may be arranged.

The chambers, at least chamber 2, are closed in an airtight manner, preferably via a door with an appropriate closing mechanism.

The temperature in the second chamber is also in a temperature range of −50° C. to 100° C., preferably in a temperature range of −30° C. to 60° C., particularly preferably in a temperature range between −10° C. and 50° C.

The pressure in the chamber 2 can also be adjustable and/or variable in a pressure range between 0.001 bar and 10 bar, in particular between 0.1 bar and 5 bar.

A plurality of first and second adapters may also be provided in this embodiment variant according to the invention.

It is particularly advantageous here that the main body parts can be pulled out of the device, whereby they can be very easily fitted with the object to be cleaned in the form of jackets, gowns and the like.

FIG. 7 shows the air flow (in the device). The air enters the third chamber 2.3 (main flow L1—through line) and passes via the inlet regulation 3.3 into the second chamber 2.3. From there this is divided into
a first air flow L4.5 (shown dashed), which leads from below into the first main body 4.5,
a second air flow L4.6 (dash-dot line), which leads from below into the second main body 4.6,
a third air flow L2 (thinner through line), which is led laterally upwards via the second chamber 2.2 and enters the first chamber 2 laterally via the apertures and ventilation slots,
a fourth air flow L4 (represented by the bold arrows) which creates a circulation of air between the first chamber 2 (main chamber/cleaning chamber), the second chamber and the fourth chamber L.4, preferably when there is no vacuum in the chamber 2 and the second chamber 2.2.

The second air flow L4.5 emerges from through-flow openings (not designated) of the first main body 4.5 after passing the UV lamp (not shown).

After flowing through the second main body 4.6, the third air flow L4.6 also exits at outlet openings in the projections V in the upper region or next to the projections V located here at the bottom in the area of the UV lamps.

As already described above, the main bodies 4 (4.5, 4.6) can be of different design and also several different ones can be integrated in one device, preferably interchangeably.

This is then implemented via an interface between main body 4, 4.5, 4.6 and cabinet body or device, wherein the interface is intended to ensure easy removal of a main body and insertion of a new main body.

The interface is preferably designed in the form of a rail system by means of which the receiving element 4 (4.5, 4.6) can also be pulled out of the device or the box/cabinet body to such an extent that it can be easily loaded with the objects to be cleaned or in order to remove the cleaned objects.

Alternatively, the air flow may enter the chamber 2 through a double lateral wall of the chamber and enter the adapters, which are rotated 90° and guided as in the previous text.

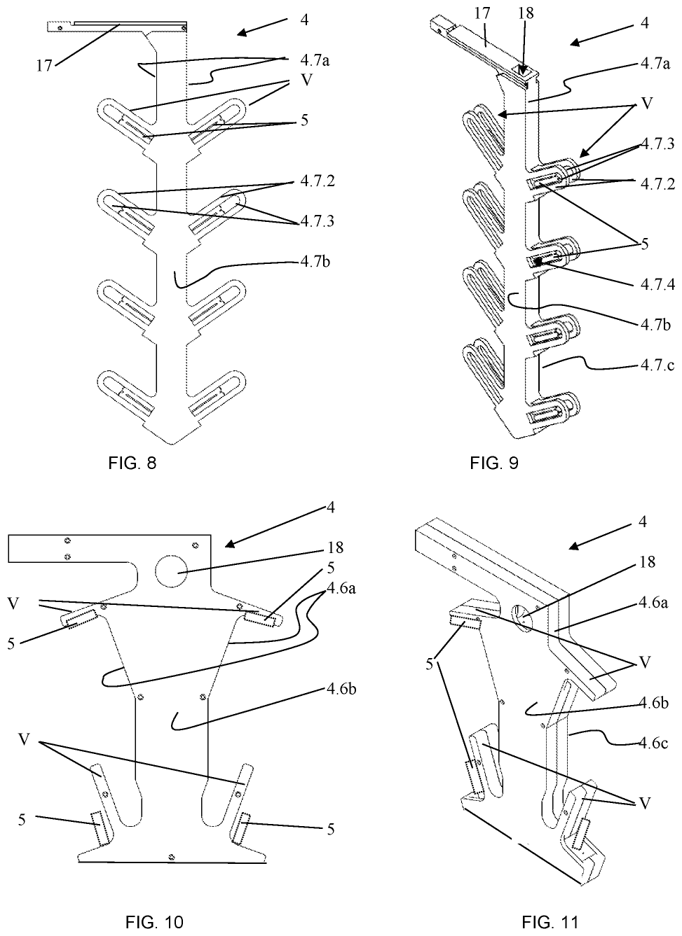

In FIGS. 8 to 11, two different main bodies 4 are shown by way of example, which have a plurality of receptacles in the form of projections V arranged opposite one another and one above the other.

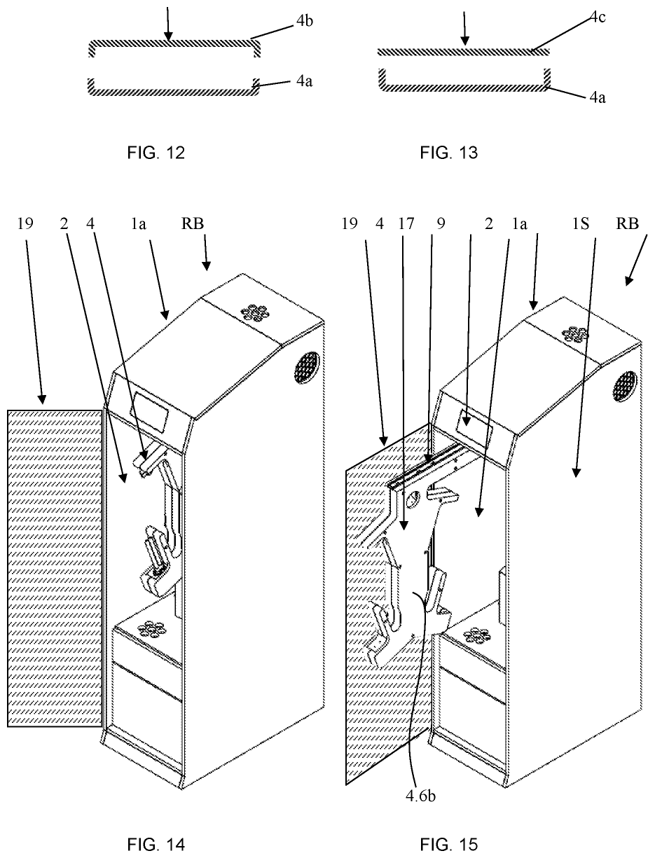

According to FIGS. 8 and 9, a hollow and vertically extending main body 4 has on opposite sides 4.7a a plurality of projections V extending laterally and obliquely upwards. The projections V are each formed here in pairs in the form of mutually parallel webs 4.7.2 (see FIG. 12). A slot-like aperture 4.7.3 is present in each web.

A through-flow opening 4.7.4 is present in the respective side 4.7.a of the main body 4 between each two webs 4.7.2 arranged in pairs. Furthermore, the UV radiation source 5, preferably a UVC lamp, is arranged between the two webs 4.7.2.

Between the two sides 4.7a a front side 4b and a rear side 4.7.c extend respectively.

Preferably, front side 4.7b and rear side 4.7c with the adjoining webs are cut from one plate and are spaced apart from each other over the sides 4.7.a.

Front side 4.7b, rear side 4.7c and sides 4.7.a are preferably made of wood, in particular plywood, and joined together—glued and/or screwed—so that the hollow body is formed in the shape of the main body 4.

On the upper side of the third adapter 4.7 there is a rail 17, via which the third adapter 4.7 can be connected to the cabinet body not shown here and can be pushed into and out of it. Furthermore, at the upper end there is an inlet opening 18 through which air flows into the third adapter 4.3 during operation.

During the cleaning process, air thus flows via the inlet opening 18 into the main body 4 into its cavity and via the through-flow openings 4.7.4 to the outside and thereby past the UV radiation sources 5 and between the projections 4.7.1 and thus the webs 4.7.2 and their openings 4.7.3. If one or more objects are placed above one or more projections V, these objects are reliably disinfected by the ozone-carrying air stream and the UVC light, optionally in conjunction with a set temperature and/or a pressure, preferably a negative pressure, in the chamber 2 (cleaning chamber) not shown here.

FIGS. 10 and 11 show a detailed diagram of a further variant of a main body 4 for one or more objects according to FIG. 7, in which the rails are not shown.

This is also designed as a hollow body and is composed of panels, in particular of wood or plastic, but preferably of plywood.

It has sides 4.6.a and a front side 4.6.b and a rear side 4.6.c which are joined together. In the front side 4.6.a and the rear side 4.6.b, apertures are arranged in the upper area as inlet openings 18.

The front side 4.6b is thereby preferably detachably connected to the sides 4.6a. Here, too, the main body 4 has projections V. The projections V adjoin the main body 4 in pairs. In this case, two projections V are arranged facing away from each other, opposite each other in the upper region, and two projections V are arranged facing away from each other, opposite each other in the lower region of the main body 4. The two upper projections V are angled slightly downward in opposition to each other so as to form a hanger-shaped receptacle for garments in the form of jackets, coats, gowns and the like. The two lower projections V project upwardly and outwardly at an angle. In the area of the projections V, the UV radiation sources 5, which are shown here as dashed lines, are arranged. Preferably, these are located within or in the region of through-flow openings of the main body not shown here in front of the projections V. In the projections V, apertures not visible here are arranged through which the air flows out.

Shoes, gloves, respiratory masks and the like can be positioned above the lower projections.

In the case of longer garments that extend over the lower projections V, the garments are also disinfected safely and reliably on/in their underside.

Cables run inside or along the outside of the main body 4, via which the power supply to the UV radiation sources 5 (preferably UVC lamps, in particular UVC LEDs) is implemented. This is also implemented in this way in the other designs of main bodies. Otherwise, the structure of the main body 4 corresponds to the design shown in FIG. 5.

Preferably, the interfaces of receiving elements to the cabinet body 1a are designed the same for the different main bodies in order to ensure their interchangeability.

In FIG. 12 it is indicated that the main body 4, which is in the form of a hollow body, can be formed from two half shells 4a, 4b.

FIG. 13 shows as a principle sketch that the main body 4 is composed of a half shell 4a and a plate 4c.

Both parts (half-shells) 4a, 4b, or 4a, 4c are joined together, e.g. non-detachably by gluing or welding or also by means of a detachable connection, in the case of plastic for example by a clip system or in the case of sheet metal e.g. by screw connections.

In this case, the main body 4 is made of plastic or sheet metal, for example, and the half-shell is made by deep drawing, for example.

If plastic is used, the receiving element could also be produced in one piece by blow molding.

FIG. 14 shows the device in the form of a box RB whose cabinet body 1a can be closed at the front by means of a door 19, in the open position of the door 19. It can be seen in the box RB a main body 4 arranged in the chamber 2.

According to FIG. 15, the main body 4 has been pulled out of the chamber 2 by means of rails 17 and can now be loaded. The front side 4.6b and the rear side of the main body 4, which is not indicated here, are aligned substantially parallel to the side walls 1S of the cabinet body 1a of the box RB. After the second main body 4 has been pushed back into the chamber 2, the door 19 is closed (preferably in an airtight manner) and the parameters required for cleaning can be set and the cleaning process started via the display 9 of the control unit. Alternatively, it is possible to use an app via a mobile device (e.g. laptop or smartphone) to set, start and stop the cleaning process and also to lock, unlock and inform the cleaning device of the status and completion of the cleaning process.

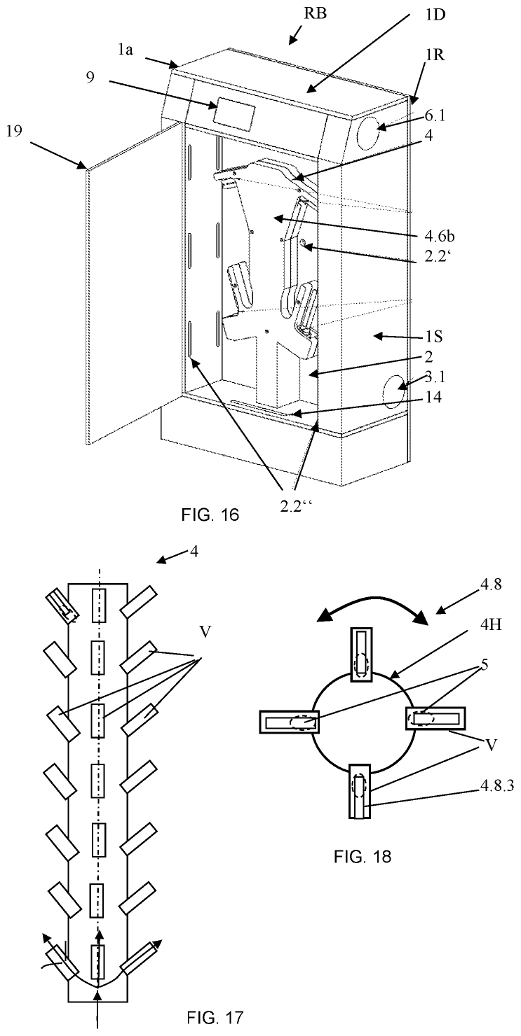

FIG. 16 shows a further variant of a box RB, in which a main body is aligned with its front side 4.6b substantially parallel to the rear wall 1R of the cabinet body 1a, in comparison with FIGS. 14 and 15. Again, the main body 4 may be replaceable and/or withdrawable from the device via an interface, although this is not shown. The box RB also has here a preferably hinged lid 1D, which allows access to exchangeable functional elements in the upper fourth chamber not designated here. Input is provided via the display 9. The front is formed by a hinged door 19. This can be made here and also in the aforementioned exemplary embodiments of UV-stable material, preferably plastic, polymer mixtures, coated glass or of metal or wood. The main body 3 is also designed as a hollow body and is preferably formed in two layers (from two half shells). In the area of the UV radiation sources, the adapter 4.6 has air outlets in the article made of textile material, which is not shown here, however.

A double-walled rear wall 1R with adapted air apertures 2.2′ to the chamber 2 is provided. In the double-walled side walls 1S there are likewise air apertures 2.2′ to the chamber 2. Furthermore, there is an additional outlet for, for example, water vapor 14 in the bottom of chamber 2.

In a side wall 1s there is an air inlet 3.1 at the bottom and an air outlet 6.1 at the top. This constructive design of the device, in particular of the cabinet body, can of course also be provided for a different design, arrangement or accommodation of the main body.

In particular, when a negative pressure is generated in the chamber 2, it is advantageous if the air can be exhausted via the double rear wall 1R the double side walls 1S via the air apertures.

It is also possible to introduce hot air into chamber 2 via the air openings 2.2″.

The generation of negative pressure in chamber 2 and the introduction of hot air into chamber 2 do not occur simultaneously.

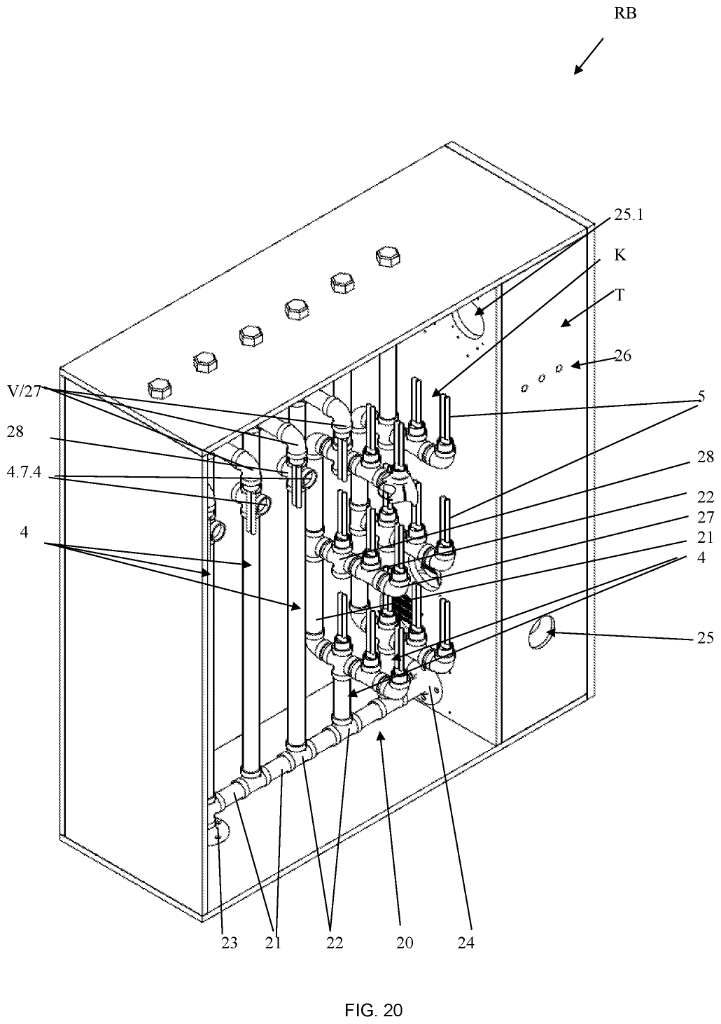

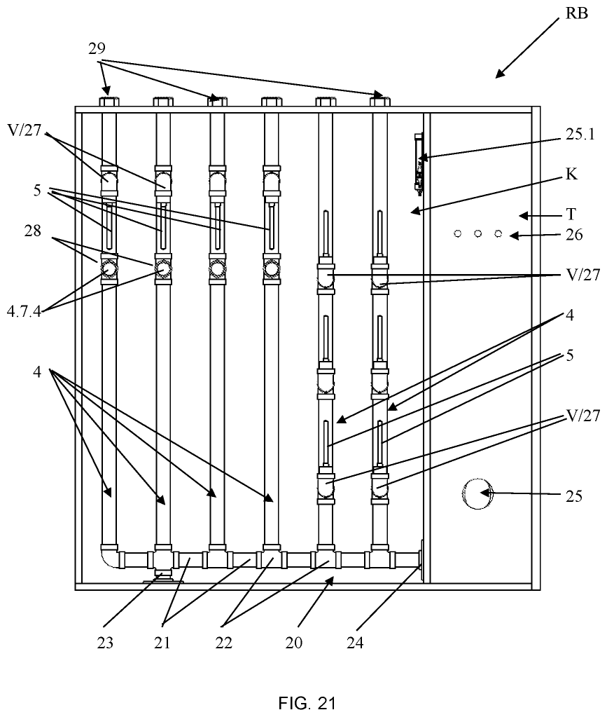

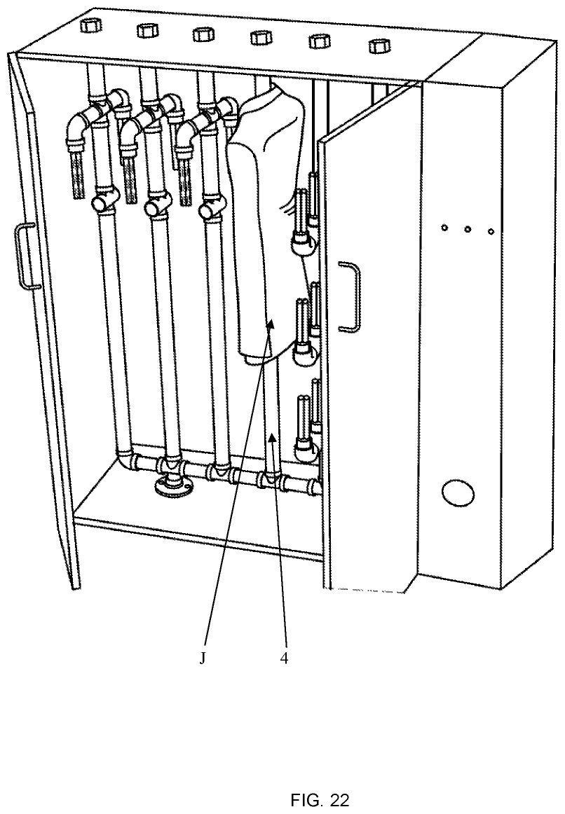

A further design of the main body 4 is provided in FIG. 20 in front view, in FIG. 21 in top view (shown enlarged) and in FIG. 22 when arranged in the chamber 2 of a box RB.

The main body 4 is formed in the shape of a tubular hollow body, on the circumference of which a plurality of projections 4V each extend in a plane, and a plurality of projections 4V are arranged one above the other. The projections V face obliquely outwardly and upwardly. Each projection 4V adjoins an aperture of the main body 4, which is not shown here. A UV radiation source 5, in particular a UVC lamp (preferably a UVC LED), is arranged within each projection V, which is indicated here by dashed lines. The projections V have openings 4.8.3 and are open at their free end.

The air supply is provided at the bottom or top of the adapter 4.8. The air flows into the main body 4 and through its through-flow openings, not visible and not designated here, into the projections V, past the UVC radiation sources 5 and through the projections V open towards the front and their openings 4.8.3.

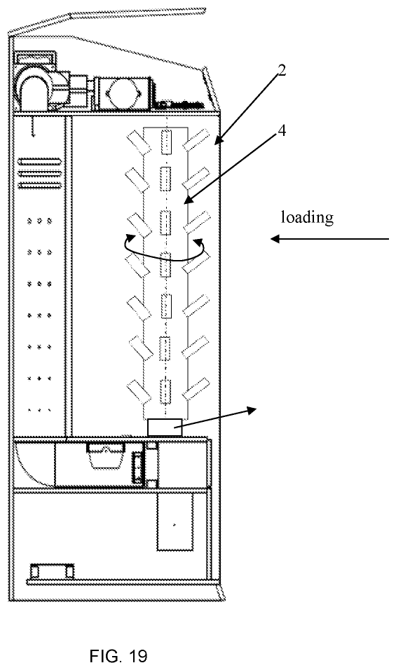

According to FIG. 19, the main body 4 arranged in the chamber 2 is rotatably mounted at its lower end for loading (here from the direction of the arrow).

It is also possible that the main body 4 also rotates during cleaning in the chamber 2. In this case, a rotary drive is operatively connected to the main body 4 and the power supply to the UV radiation sources must also be ensured during rotation (e.g. via sliding contacts or via cables and a back and forth rotation with a rotation angle of less than 360°).

By rotating the main body 4 during cleaning, faster drying, better removal of dirt and better removal of allergy-causing pollen can be realized.

Especially when cleaning shoes, the centrifugal force occurring during the rotation promotes the preservation of the shoe shape.

Here, too, as an alternative or in addition to the rotatable mounting in the chamber, the extraction can be provided by means of a rail system, which may also be arranged at the bottom of the receiving element.

In the presented embodiment variants, the projections V can be formed integrally with the main body of the receiving element or can be detachably or non-detachably connected to the receiving element. A detachable connection enables the projections to be replaced.

FIGS. 20 and 21 show a device in the form of a box RB with a cleaning chamber K in which a plurality of tubular main bodies 4 are arranged, which are arranged next to one another and extend vertically upwards and, here by way of example, are connected to one another at their lower ends by a connecting line 20.

Next to the cleaning chamber K is a technical chamber T, in which all technical components such as control unit, pump(s), filter and the like are arranged.

The connecting pipe 20 is composed of a plurality of pipe sections 21 and T-shaped fittings 22 (pipe connectors). The T-shaped fittings 22 have connections on both sides to the pipe sections 21 and a connection, facing upwards here, to a respective tubular main body 4. At its end facing away from the technical chamber, the connecting pipe 20 is supported by means of a foot 23 at the bottom on the base of the box RB, which is not designated, and in the direction towards the technical chamber T, the connecting pipe 20 is connected to the partition wall to the technical chamber T, which is not designated, via a connecting flange 24. In the region of the connecting flange 24, an aperture is arranged in the partition wall, so that a pump and/or vacuum pump (not shown) is connected to the connecting pipe 20, so that air flows into the connecting pipe under pressure via the pump(s) or air is extracted. In the technical chamber T there is an aperture 25 through which air can flow in or out. Furthermore, an air flow inlet 25.1 leads from the technical chamber T through the partition wall to the cleaning chamber K, which is not designated.

Control knobs 26 are provided in the front of the technical chamber T.

Three tubular main bodies 4, shown here on the left, extend substantially continuously upwardly from the connecting pipe 20. Each of the three main bodies 4 shown here on the left has, in the upper region in the direction of the front and in the direction of the rear of the box RB, downwardly angled projections V in the form of 90° angle pieces 27, into which UVC lamp means 5 are screwed so as to project downwardly. On these main bodies, for example, jackets or gowns can be hung. Somewhat below the UVC lamps 5, there are further flow-through openings 4.7.4 in the tubular main body 4, which are formed in cross-shaped fittings 28. Each cross-shaped fitting 28 has four connections. A lower and an upper fitting are connected to vertically extending pipes of the main body, the second lateral fittings are open so that air can flow out of them.

Garments such as jackets or gowns can be hung over the left-hand main bodies 4 with their projections V, for example.

Two tubular main bodies 4 are shown on the right, consisting of shorter pipe sections 21 connected to each other. Three rows of horizontal pipe elements of T-shaped fittings 22, 90° elbow fittings 27 (angled upwards) and cross-shaped fittings 28 each with three upwardly facing projections V with undesignated through-flow openings, into which UVC lamps 5 are screwed, extend from vertical tubular pieces 21. These serve, for example, to receive smaller objects such as breathing masks and the like. The doors are not shown in FIGS. 20 and 21.

FIG. 22 shows the device according to FIGS. 20 and 21 from a different perspective with undesignated doors and an exemplary gown J or jacket positioned above the main body 4.

It is of course possible to arrange more or less of the main bodies in a box and to design the structure of the main bodies and the number and orientation of the projections V with the UVC lamps as desired.

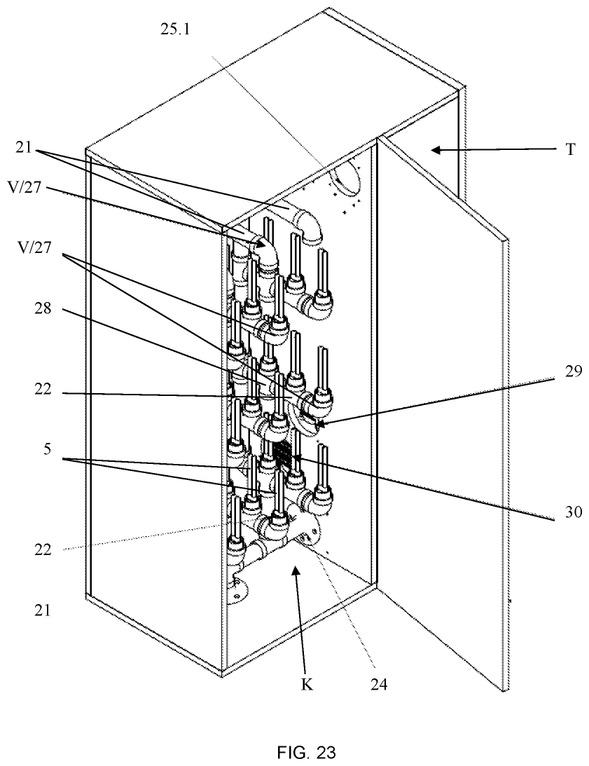

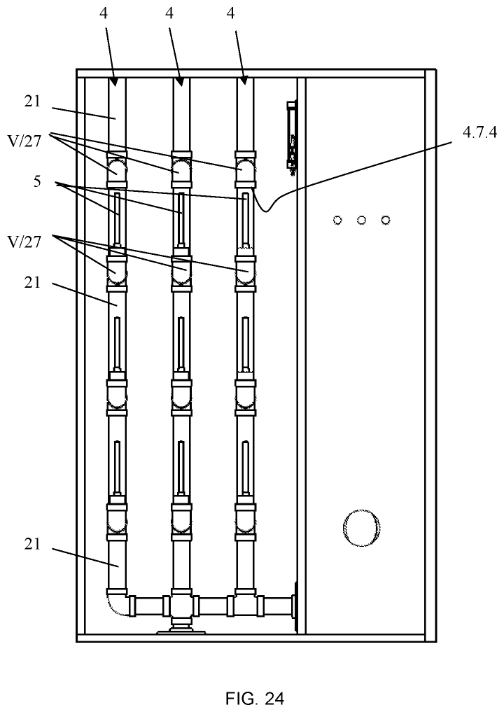

FIGS. 23 and 24 show a device which is substantially as in FIGS. 20 to 22, but with only three main bodies 4 which have projections V arranged one above the other and side by side and facing upwards, like the two right-hand main bodies 4 shown in FIGS. 20 to 22.

Also in the variant according to FIGS. 23 and 24, the main bodies 4 are composed of several pipe sections and pipe connecting elements. These are pipe sections 21, T-shaped fittings 22, 90° angle pieces 27 and cross-shaped fittings 28. The 90° angle pieces 27, which are angled upwards, form upwardly facing projections V into which the UVC lamps 5 are screwed.

Above the UVC lamps 5 located at the top, additional angle pieces 27 are arranged here, the flow openings 4.7.4 of which face downwards.

The other angle pieces 27, which support the UVC lamps 5, also have unmarked through-flow openings in the direction of the UVC lamps 5. In the intermediate wall (not designated) between the cleaning chamber K and the technical chamber T there are an air flow inlet 25.1, a filter battery and an exhaust air system 29 as well as a circulating air generator and heating element 30 (see FIG. 23).

Again, the number and design of the main bodies 4 and the number, design and orientation of the projections V and the number and orientation of the UVC lamps 4 can be designed to suit individual requirements.

In FIG. 25, a device is shown which has only a cross-shaped hollow main body 4 formed by a cross-shaped fitting 28. There are three upwardly facing projections V. The left and right projections V are each formed by angled pieces 27 screwed into lateral connections of the main body 4 and angled upwardly. The middle projection V is formed by the upwardly facing connection of the main body 4. The upwardly facing projections V of these pipe connecting elements have through-flow openings 4.7.4. In these through-flow openings 4.7.4 adapter elements 31 are inserted, which accommodate the UVC lamps 5 and which are provided with slots 32 through which the air flow can exit to flow past the UVC lamps. The UVC lamps 5 are arranged in the three adapter elements 31 of the projections V.

An aperture 25 is arranged in a side wall of the box RB, which forms an air flow inlet. A recirculation channel 33 opens into the intermediate wall to a technical chamber not shown here on the rear side.

The main body V is attached by means of an angle piece 27, which adjoins the downwardly facing connection of the cross-shaped fitting 27 and is attached to the rear wall of the box RB by means of a connecting flange 24. In the rear wall, an aperture not visible here is provided in this area, through which air flows into the main body 4, which flows past the UVC lamps 5 via the projections V and the adapter elements 31. Only a control knob 26 for switching on and off is provided on this device. The box RB can be closed with an undesignated door during the cleaning process.

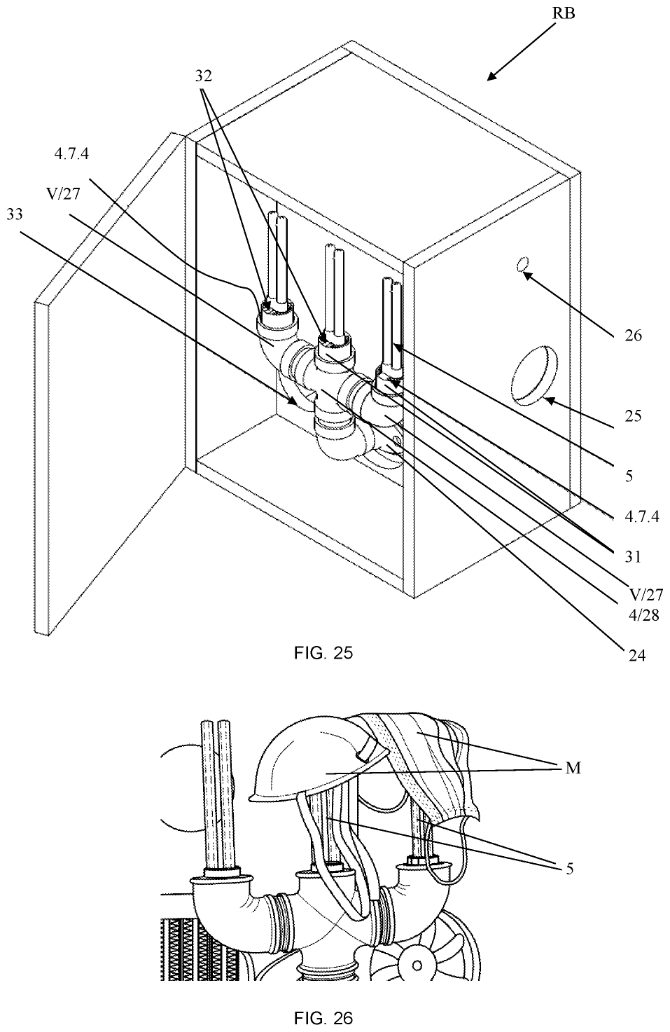

FIG. 26 shows a partial view of the device according to FIG. 25 with objects to be cleaned in the form of breathing masks M received by two UVC lamps 5.

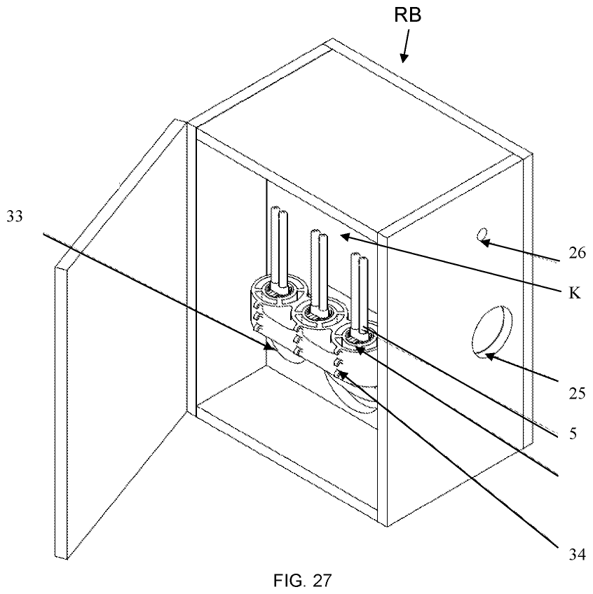

FIG. 27 also shows a main body 4 arranged in a box RB, which has three upwardly directed short projections V with through-flow openings 4.7.4 and adapter elements 31 with slots 32 seated therein. The main body 4 has been produced here, for example, by 3D printing or injection molding.

Here, too, the UVC lamps 5 facing upwards are fixed in the adapter elements 31, above which, for example, breathing masks can be positioned.

Several hooks 34 are provided at the front of the main body, on which objects to be cleaned, such as respirator masks, can be accommodated.

The projections V have through-flow openings 4.7.1 to the individual adapter elements 31 on their upper side.

An aperture 25 is provided in a side wall, which forms an air flow inlet, and a recirculation channel 33 opens into the rear wall of the cleaning chamber K. An undesignated lockable door is also provided. There is also only one control knob for starting and stopping the cleaning process.

It is also possible that the control button is only used to start the cleaning process and that this process is automatically ended after a certain time.

The adapter elements used in FIGS. 25 and 27 can also be used in the other solutions described above, in particular in the variants illustrated in FIGS. 17 to 24.

In particular, due to the modular design, which is possible by using pipes, pipe sections, pipe connecting elements, the devices can be designed variably with regard to the number and size of the main bodies, the number and arrangement of the UVC lamps.

It is possible to provide a modular system with which the device can be adapted to individual requirements.

The solution according to the invention enables for the first time a versatile possibility of accommodating a wide variety of objects to be cleaned in a corresponding device, as well as their efficient cleaning and elimination of pathogens.

### LIST OF REFERENCE NUMERALS

RB Box
1 Housing
1a Body
1B Base
1D Lid
1R Rear wall
1S Side wall
2 Chamber
2.2 Second chamber
2.3 Third chamber
2.4 Fourth chamber
2.2′ Apertures
2.2″ Air apertures
3 Air supply
3′ Inlet regulation
3.1 Air inlet
3.2 Fan
3.3 Chamber-side end of the air supply
3.4 Heating element
4 Main body
4.1 Hook
4.2 Carrier element
4.3 Carrier element
4.4 Connection
4.5 First adapter/main body
4.5.1 Air guide
4.5.2 Outlet opening
4.6 Second adapter/main body
4.6′ Receiving element
4.6.1 Air guide
6.6.2 Outlet opening/aperture
4.6a Sides
4.6b Front side
4.6c Rear side
4.7a Sides
4.7.2 Webs
4.7.3 Apertures
4.7.4 Through-flow openings
4.8.3 Breakthroughs
4a, 4b Half shells
4c Plate
4A Vertically extending region
5 UV radiation source
6 Suction device
6.1 Air outlet
6.1.1 Outlet openings
6.2 Fan
7 Water tank
7.1 Heating element
7.2 Conduit
7.3 Evaporator
8 Fragrance capsule
8.1 Manual controller
9 Control unit with display
10 Shoe/protective equipment
11 Pressure system
12 Cross-struts
13 Suspension
14 Openings
14.2 Openings
15 Connection
16 Openings
17 Rail
18 Inlet opening
19 Door
20 Connecting line
21 Pipe section
22 T-shaped fitting
23 Foot
24 Connecting flange
25 Aperture
25.1 Air flow inlet
26 Control knob/control buttons
27 90° angle piece
28 Cross-shaped fitting
29 Filter battery and exhaust system
30 Circulating air generators and heating elements
31 Adapter elements
32 Slots
33 Recirculating air channel
34 Hook
A1, A2 Covers
B Base
D Lid
J Gown/jacket
L1 First air flow
L4.6 Second air flow
L2 Third air flow
L4 Fourth air flow
M Breathing masks
S1, S2 Side parts
V Projection
W1 Horizontal wall between first and second chamber
W2 Vertical wall between first and second chamber
W3 Horizontal wall
W4 Horizontal wall

1. Device for cleaning and disinfecting at least one object such as clothing and/or protective masks and/or personal protective equipment, in particular for the medical or laboratory sector, wherein the device has a chamber which is surrounded by a housing and in which at least one main body is arranged for receiving the object, characterized in that the main body has a cavity and, on its outer circumference, one or more through-flow openings to the cavity, wherein at least one UV radiation source which emits UV light and generates ozone is arranged in the region of at least one through-flow opening, and in that at least one air supply leads into the cavity of the main body, wherein the air can be conducted through the through-flow opening past the UV radiation source into the object.

2. Device according to claim 1, characterized in that the UV-C radiation source adjoins or is adjacent to a through-flow opening of the main body or at least partially engages in the through-flow opening or is arranged adjacent to a through-flow opening.

3. Device according to claim 1, characterized in that the main body has a plurality of projections, wherein one projection adjoins in each case a through-flow opening or is arranged adjacent to a through-flow opening.

4. Device according to claim 3, characterized in that the projections adjoin the outer periphery of the main body.

5. Device according to claim 3, characterized in that projections extend horizontally or vertically and/or that projections are angled upwards or downwards and a UVC radiation source can be screwed into one, several or all projections.

6. Device according to claim 1, characterized in that the main body extends vertically or horizontally in the chamber and in that the air supply leads into the main body through a lower opening and/or an upper opening and/or a lateral opening.

7. Device according to claim 1, characterized in that a plurality of through-flow openings and projections are arranged circumferentially and/or one above the other on the main body, wherein the main body and/or the projections have an angular or round cross-section.

8. Device according to claim 1, characterized in that the main body can be pulled out of the device and pushed in by means of a rail system.

9. Device according to claim 1, characterized in that the main body is rotatably mounted in the device.

10. Device according to claim 1, characterized in that a negative pressure and/or a positive pressure can be generated in the chamber.

11. Device according to claim 1, characterized in that the air which can be supplied to the main body has a temperature between −30 C and 150° C.

12. Device according to claim 1, characterized in that the device comprises:
A) means for adjusting the pressure in the chamber and/or
B) means for adjusting the temperature in the chamber,
as well as an air supply leading from outside the housing into the interior of the chamber with an air inlet and with at least one air outlet leading from the chamber out of the housing, wherein the supplied air can be conducted via the hollow main body and the UV radiation source(s) into the interior of the objects, in particular clothing and/or masks.

13. Device according to claim 1, characterized in that the main body is interchangeable and/or in that a plurality of main bodies can be arranged within a chamber,

14. Device according to claim 1, characterized in that a plurality of main bodies are interconnected and have a common air supply.

15. Device according to claim 1, characterized in that the cleaning cycle is determinable or adjustable by means of a control unit and the parameters such as temperature and/or pressure and/or addition of fragrances and/or silver nanoparticles are determinable or adjustable.

16. Device according to claim 1, characterized in that at least one fragrance element is arranged in the air supply.

17. Device according to claim 1, characterized in that the device comprises a water tank with a heating element such that water vapor can be generated and that the water vapor can be supplied to the air supply via a channel.

18. Method for cleaning and disinfecting at least one object, in particular at least one object such as clothing and/or protective masks and/or personal protective equipment, for the medical, veterinary or laboratory sector with a device according to claim 1, characterized in that the object is received in the chamber by means of a receiving element and UV-C light in the non-visible wavelength range and ozone are generated in the chamber during a cleaning cycle for removing pathogens by means of at least one UV light source attached to the receiving element, and in that the chamber 2 is subjected to a negative pressure and/or a positive pressure, wherein an air flow is generated through the hollow receiving element by means of an air inlet leading from the housing into the chamber and an air outlet leading from the chamber out of the housing, and the air flows via outlets on the hollow receiving element and flows past ozone-generating UV radiation sources and is guided into the object.

19. Method according to claim 18, characterized in that the pressure is adjustable and/or variable in a pressure range from 0.1 bar to 5 bar.

20. Method according to claim 18, characterized in that the temperature in the chamber is adjustable and/or variable in a temperature range from −30° C. to 150° C.

21. Method according to claim 18, characterized in that a negative pressure is alternately generated during a cleaning cycle or in that the pressure alternates between a negative pressure and a positive pressure.

22. Method according to claim 18 for killing bacteria, viruses, fungi and spores thereof.

23. Method according to claim 18, characterized in that during a cleaning cycle the temperature is alternately reduced from 150° C. to −30° C. within 0.5 to 66 minutes and then increased again.

24. Method according to claim 18, characterized in that during a cleaning cycle the pressure is alternately reduced from 5 bar to 0.1 bar within 0.1 to 66 minutes and then increased again.

25. Method according to claim 18, characterized in that in particular protective equipment and clothing as well as laboratory objects made of typical textile or chemical polymers and/or polymer fibers are cleaned and disinfected in a short time, in particular within 1 to 100 minutes and preferably within 1 to 30 minutes.

26. (canceled)

27. (canceled)

28. Objects, in particular medical devices and objects and medical protective clothing, treated in a device according to claim 1 and with a method according to claim 18, characterized in that their surfaces at the end of a respective treatment have only residues of >less than =10%, preferably less than or equal to 1%, in particular less than or equal to 0.1%, of biochemically biologically active organisms and/or biochemical materials.

29. Objects according to claim 28, characterized in that the objects are intended for single use only but are reusable after treatment.
